(19) United States
(12) Patent Application Publication
(10) Pub. No.: US 2026/0289784 A1
(43) Pub. Date: Sep. 24, 2026

(54) METHODS, SYSTEMS, AND STORAGE MEDIA FOR EVALUATING HEMODYNAMIC CHARACTERISTICS

(71) Applicant: SHANGHAI UNITED IMAGING HEALTHCARE CO., LTD., Shanghai (CN)

(72) Inventor: Xiaodong WANG, Shanghai (CN)

(73) Assignee: SHANGHAI UNITED IMAGING HEALTHCARE CO., LTD., Shanghai (CN)

(21) Appl. No.: 19/678,432

(22) Filed: May 15, 2026

Related U.S. Application Data
(63) Continuation of application No. PCT/CN2024/122239, filed on Sep. 29, 2024.

(30) Foreign Application Priority Data
Dec. 15, 2023 (CN) 202311733395.1

(51) Int. Cl. G06T 7/00; A61B 6/00; A61B 6/50; G06T 7/246; G06T 7/62; G06T 17/20

(52) U.S. Cl. CPC G06T 7/0014; A61B 6/507; A61B 6/5217; G06T 7/251; G06T 7/62; G06T 17/20; G06T 2207/20036; G06T 2207/20081; G06T 2207/20084; G06T 2207/30104; G06T 2207/30172; G06T 2210/41

(57) ABSTRACT

The present disclosure provides a method for evaluating a hemodynamic characteristic, comprising: obtaining a medical image of a subject, the medical image including at least a portion of a vascular structure; determining at least one property parameter of the vascular structure by inputting the medical image into a trained parameter determination model; and determining at least one hemodynamic characteristic of the vascular structure by inputting the at least one property parameter into a vascular fluid dynamic model.

### CROSS-REFERENCE TO RELATED APPLICATIONS

This application is a continuation of International Application No. PCT/CN2024/122239, filed on Sep. 29, 2024, which claims priority to Chinese Patent Application No. CN 202311733395.1, filed on Dec. 15, 2023, the entire contents of which are hereby incorporated by reference.

### TECHNICAL FIELD

The present disclosure relates to the field of medical technology, particularly to a method, a system, and a storage medium for evaluating a hemodynamic characteristic.

### BACKGROUND

With the development of medical technology, medical imaging of human vascular structures has become increasingly accurate. Based on the medical imaging of human vascular structures, the evaluation of hemodynamic in these structures has also become increasingly important.

Traditional methods for hemodynamic evaluation may include generating a three-dimensional vascular model and using computational fluid dynamics (CFD). Due to the complexity of constructing the three-dimensional model, a one-dimensional model may be used to evaluate a hemodynamic characteristic with CFD. However, a traditional method of combining the one-dimensional model with CFD for hemodynamic evaluation have issues of inaccurate results in hemodynamic characteristic evaluation.

Therefore, there is a need to propose a method, a system, and a storage medium for evaluating a hemodynamic characteristic to improve the accuracy and speed in hemodynamic evaluation using a one-dimensional model.

### SUMMARY

One or more embodiments of the present disclosure provide a method for evaluating a hemodynamic characteristic implemented on a device including at least one processing device and at least one storage device. The method may include: obtaining a medical image of an object, the medical image including at least a portion of a vascular structure. The method may include: determining at least one property parameter of the vascular structure by inputting the medical image into a trained parameter determination model. The method may include: determining at least one hemodynamic characteristic of the vascular structure by inputting the at least one property parameter into a vascular fluid dynamic model.

One or more embodiments of the present disclosure provide a system for evaluating a hemodynamic characteristic. The system may include at least one storage device including a set of instructions. The system may include at least one processor in communication with the at least one storage device, wherein when executing the set of instructions, the at least one processor is directed to: obtain a medical image of an object, the medical image including at least a portion of a vascular structure; determine at least one property parameter of the vascular structure by inputting the medical image into a trained parameter determination model; and determine at least one hemodynamic characteristic of the vascular structure by inputting the at least one property parameter into a vascular fluid dynamic model for a hemodynamic characteristic evaluation.

One or more embodiments of the present disclosure provide a system for evaluating a hemodynamic characteristic. The system may include an acquisition module configured to obtain a medical image of an object, the medical image including at least a portion of a vascular structure. The system may include a recognition module configured to determine at least one property parameter of the vascular structure by inputting the medical image into a trained parameter determination model. The system may include an evaluation module configured to obtain at least one hemodynamic characteristic of the vascular structure by inputting the at least one property parameter into a vascular fluid dynamic model for a hemodynamic characteristic evaluation.

One or more embodiments of the present disclosure provide a non-transitory computer readable medium. The non-transitory computer readable medium may include at least one set of instructions, wherein when executed by one or more processors of a computing device, the at least one set of instructions causes the computing device to perform a method for evaluating a hemodynamic characteristic. The method may include: obtaining a medical image of an object, the medical image including at least a portion of a vascular structure. The method may include: determining at least one property parameter of the vascular structure by inputting the medical image into a trained parameter determination model. The method may include: determining at least one hemodynamic characteristic of the vascular structure by inputting the at least one property parameter into a vascular fluid dynamic model for a hemodynamic characteristic evaluation.

One or more embodiments of the present disclosure provide a method for training a parameter determination model. The method may be implemented on a device including at least one processing device and at least one storage device. The parameter determination model is configured to determine a property parameter of a vascular structure in a medical image, and the property parameter is configured to determine a hemodynamic characteristic of the vascular structure based on a vascular fluid dynamic model. The method may include: obtaining a first medical image sample and a corresponding gold standard hemodynamic characteristic. The method may include: determining a parameter determination result by inputting the first medical image sample into an initial parameter determination model. The method may include: obtaining the trained parameter determination model based on the gold standard hemodynamic characteristic and the parameter determination result.

One or more embodiments of the present disclosure provide a system for training a parameter determination model, wherein the parameter determination model is configured to determine a property parameter of a vascular structure in a medical image, and the property parameter is configured to determine a hemodynamic characteristic of the vascular structure based on a vascular fluid dynamic model. The system may include at least one storage device including a set of instructions. The system may include at least one processor in communication with the at least one storage device, wherein when executing the set of instructions, the at least one processor is directed to: obtain a first medical image sample and a corresponding gold standard hemodynamic characteristic; determine a parameter determination result by inputting the first medical image sample into an initial parameter determination model; and obtain the trained parameter determination model on the gold standard hemodynamic characteristic and the parameter determination result.

One or more embodiments of the present disclosure provide a system for training a parameter determination model, wherein the parameter determination model is configured to determine a property parameter of a vascular structure in a medical image, and the property parameter is configured to determine a hemodynamic characteristic of the vascular structure based on a vascular fluid dynamic model. The system may include a training module configured to: obtain a first medical image sample and a corresponding gold standard hemodynamic characteristic; determine a parameter determination result by inputting the first medical image sample into an initial parameter determination model; and obtain the trained parameter determination model based on the gold standard hemodynamic characteristic and the parameter determination result.

One or more embodiments of the present disclosure provide a non-transitory computer readable medium. The non-transitory computer readable medium may include at least one set of instructions, wherein when executed by one or more processors of a computing device, the at least one set of instructions causes the computing device to perform a method for evaluating a hemodynamic characteristic. The parameter determination model is configured to determine a property parameter of a vascular structure in a medical image, and the property parameter is configured to determine a hemodynamic characteristic of the vascular structure based on a vascular fluid dynamic model. The method may include: obtaining a first medical image sample and a corresponding gold standard hemodynamic characteristic. The method may include: determining a parameter determination result by inputting the first medical image sample into an initial parameter determination model. The method may include: obtaining the trained parameter determination model based on the gold standard hemodynamic characteristic and the parameter determination result.

### BRIEF DESCRIPTION OF THE DRAWINGS

The description will be further explained in the form of exemplary embodiments, which will be described in detail by means of accompanying drawings. These embodiments are not restrictive, in which the same numbering indicates the same structure, wherein:

FIG. 1 is a schematic diagram illustrating an application scenario of an exemplary system for evaluating a hemodynamic characteristic according to some embodiments of the present disclosure;

FIG. 2 is a schematic diagram illustrating hardware and software components of an exemplary computing device according to some embodiments of the present disclosure;

FIG. 3 is a block diagram illustrating an exemplary system for evaluating a hemodynamic characteristic according to some embodiments of the present disclosure;

FIG. 4 is a flowchart illustrating an exemplary process for evaluating a hemodynamic characteristic according to some embodiments of the present disclosure;

FIG. 5 is a schematic diagram illustrating an exemplary process for evaluating a hemodynamic characteristic according to some embodiments of the present disclosure;

FIG. 6 is a flowchart illustrating an exemplary process for training a parameter determination model according to some embodiments of the present disclosure;

FIG. 7 is a flowchart illustrating an exemplary process for obtaining a gold standard hemodynamic characteristic according to some embodiments of the present disclosure;

FIG. 8 is a flowchart illustrating an exemplary process for determining a gold standard hemodynamic characteristic according to other embodiments of the present disclosure;

FIG. 9 is a schematic diagram illustrating an exemplary process for training a parameter determination model according to some embodiments of the present disclosure;

FIG. 10 is a schematic diagram illustrating an exemplary parameter determination model according to some embodiments of the present disclosure;

FIG. 11 is a schematic diagram illustrating an exemplary medical image according to some embodiments of the present disclosure.

### DETAILED DESCRIPTION

The technical schemes of embodiments of the present disclosure will be more clearly described below, and the accompanying drawings need to be configured in the description of the embodiments will be briefly described below. Obviously, the drawings in the following description are merely some examples or embodiments of the present disclosure, and will be applied to other similar scenarios according to these accompanying drawings without paying creative labor. Unless obviously obtained from the context or the context illustrates otherwise, the same numeral in the drawings refers to the same structure or operation.

It should be understood that the “system”, “device”, “unit” and/or “module” used herein is a method for distinguishing different components, elements, components, parts or assemblies of different levels. However, if other words may achieve the same purpose, the words may be replaced by other expressions.

As shown in the present disclosure and claims, unless the context clearly prompts the exception, “a”, “one”, and/or “the” is not specifically singular, and the plural may be included. It will be further understood that the terms “comprise,” “comprises,” and/or “comprising,” “include,” “includes,” and/or “including,” when used in present disclosure, specify the presence of stated features, integers, steps, operations, elements, and/or components, but do not preclude the presence or addition of one or more other features, integers, steps, operations, elements, components, and/or groups thereof.

The flowcharts are used in present disclosure to illustrate the operations performed by the system according to the embodiment of the present disclosure. It should be understood that the preceding or following operations is not necessarily performed in order to accurately. Instead, the operations may be processed in reverse order or simultaneously. Moreover, one or more other operations may be added to the flowcharts. One or more operations may be removed from the flowcharts.

In recent years, with the development of imaging technologies such as computed tomography (CT), magnetic resonance (MR), and digital subtraction angiography (DSA), the acquisition of human vascular structures has become increasingly accurate, and functional studies based on vascular structures have also gained increasing importance, especially blood flow evaluation research based on the structure of the human cardio vascular system.

Traditional methods for evaluating a blood flow are based on computational fluid dynamics (CFD) that involves establishing a three-dimensional (3D) vascular model, meshing the 3D vascular model, determining one or more boundary conditions such as a pressure and a flow rate, solving the discretized differential equations based on the Navier-Stokes (N-S) equation, and ultimately obtaining blood flow information e.g., a blood pressure and a flow velocity within the 3D space.

However, due to the complexity of constructing the 3D vascular model and performing CFD calculations, methods for evaluating a blood flow based on reduced-order and/or dimensionality models have emerged. These methods include replacing the 3D vascular model with a one-dimensional (1D) vascular model, determining a relevant vascular parameter (e.g., a vascular diameter) based on the vascular structure contained in the 3D model, applying the relevant vascular parameter to the 1D model to indirectly obtain information from the 3D model, and then determining 1D blood flow information using fluid dynamics formulas for the 1D model.

In some embodiments, the 1D vascular model for blood flow evaluation may be constructed based on the hydraulic diameter. Using the 1D vascular model constructed based on the hydraulic diameter, the hydraulic diameter, rather than the actual diameter, may be used to determine the 3D vascular diameter, ensuring that vessels with the same hydraulic diameter have the same Reynolds number. The hydraulic diameter, introduced in fluid mechanics, is mainly used for non-circular pipe flows to assign an appropriate characteristic length for determining the Reynolds number. The hydraulic diameter may be used to make a non-circular flow channel equivalent to a circular pipe in terms of flow characteristics (mainly flow resistance characteristics). The hydraulic diameter may be defined as a ratio of a cross-sectional area of a vessel to the perimeter of the vessel at a given cross-section. For an abnormal position of a vessel (e.g., stenosis, occlusion, aneurysms, vascular wall calcification), the actual diameter cannot be obtained directly from one or more images. In such cases, the hydraulic diameter may be used as a substitute to be inputted into a 1D fluid dynamics formula to determine blood flow information, such as a blood pressure, at the stenosis site.

However, using the hydraulic diameter to determine blood flow information may only ensure that the friction force and resistance experienced by blood flowing through vessels with the same hydraulic diameter are the same, but not guarantee the same pressure drop. Therefore, the blood pressure evaluated based on the hydraulic diameter is inaccurate. Additionally, determining the cross-sectional area and the perimeter of a cross-section of the vessel at the abnormal position is not easy, and it is difficult to ensure the accuracy of the hydraulic diameter calculation.

Furthermore, there are also methods for evaluating a blood flow based on a deep neural network technique, which directly determines fluid dynamics parameters using one or more neural networks. These methods do not require fluid dynamics models at all, but fluid dynamics is a complex physical process, and vascular structure and case characteristics are highly diverse. It is unclear whether neural network training without considering fluid dynamics models can adapt to various complex vascular structures.

Based on above, the embodiments of the present disclosure provides a method for evaluating a hemodynamic characteristic with improved efficiency and accuracy. The method may include using a 1D fluid dynamics model to evaluate the hemodynamic characteristic based on an equivalent diameter. The method may use an equivalent diameter to replace the traditional hydraulic diameter and equivalent 3D vascular structures like the heart and brain into a 1D vascular fluid dynamics model. In some embodiments, the equivalent diameter is a calculated value, and is not a diameter of a real vascular interface. In some embodiments, the equivalent diameter may be input into the 1D fluid dynamics model for hemodynamic characteristic determination. The method simplifies the hemodynamic calculation process, improves calculation speed, and is of significant importance in image-based vascular evaluation system software.

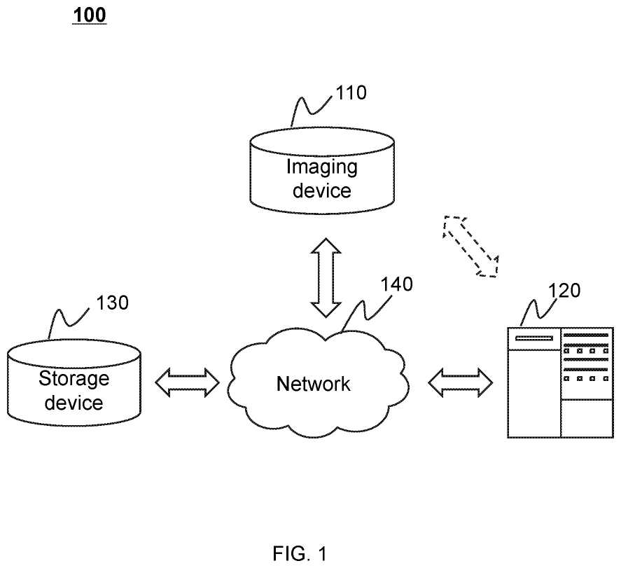

The method for evaluating a hemodynamic characteristic provided in some embodiments of the present disclosure may be applied to an application scenario as illustrated in FIG. 1.

FIG. 1 is a schematic diagram illustrating an application scenario of an exemplary system for evaluating a hemodynamic characteristic according to some embodiments of the present disclosure.

As shown in FIG. 1, the application scenario 100 of the system for evaluating a hemodynamic characteristic may include an imaging device 110, a processor 120, a storage device 130, and a network 140.

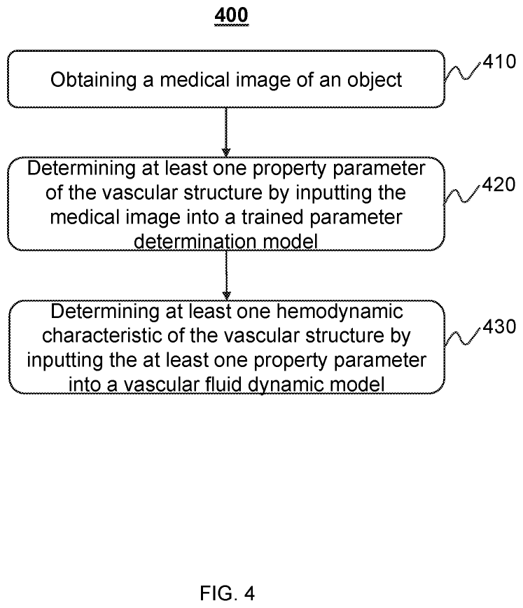

The imaging device 110 may scan a subject to obtain a medical image of a subject. For more information about the subject, please refer to FIG. 4 and its related descriptions.

The imaging device 110 may include a single modality imaging device and/or a multi-modality imaging device. The single modality imaging device may include, for example, a computed tomography (CT) device, a positron emission computed tomography (PET) device, a magnetic resonance imaging (MRI) device, an ultrasonic imaging device, an X-ray imaging device, a single photon emission computed tomography (SPECT) device, etc. The multi-modality imaging device may include, for example, an MRI-CT device, a PET-MRI device, a SPECT-MRI device, a digital subtraction angiography-magnetic resonance imaging (DSA-MRI) device, a PET-CT device, a SPECT-CT device, etc.

In some embodiments, the processor 120 and the storage device 130 may be part of the imaging device 110. In some embodiments, the imaging device 110 may send image data of a subject to the processor 120 and the storage device 130 via the network 140 for further processing.

The processor 120 may process data and/or information obtained and/or extracted from the imaging device 110, the storage device 130, and/or other storage devices. In some embodiments, the processor 120 may obtain a medical image of the subject and a trained parameter determination model. Furthermore, the processor 120 may process the medical image of the subject based on the trained parameter determination model to determine at least one property parameter of a vascular structure contained in the medical image.

In some embodiments, the processor 120 may be a single server or a server group. The server group may be centralized or distributed. In some embodiments, the processor 120 may be local or remote. For example, the processor 120 may access information and/or data stored in the imaging device 110 and/or the storage device 130 via the network 140. As another example, the processor 120 may directly connect to the imaging device 110 and/or the storage device 130 to access their stored information and/or data. In some embodiments, the processor 120 may be implemented on a cloud platform. By way of example only, the cloud platform may include a private cloud, a public cloud, a hybrid cloud, a community cloud, a distributed cloud, an internal cloud, a multi-layer cloud, or any combination thereof.

In some embodiments, the processor 120 may be implemented on a computer device. The computer device may be a computer connected to the imaging device 110, such as a laptop or a desktop computer placed in a scanning room or an operation room. In some embodiments, the processor 120 may be included in the imaging device 110 and/or other possible system components, for example, the processor 120 or a module capable of realizing the functions of the processor 120 may be integrated into the imaging device 110 and/or other possible system components.

The storage device 130 may be configured to store data, instructions, and/or any other information. In some embodiments, the storage device 130 may store data obtained from the imaging device 110 and/or the processor 120. For example, the storage device 130 may store image data, a trained parameter determination model, and related data. In some embodiments, the storage device 130 may store data and/or instructions used by the processor 120 to execute or utilize to perform the exemplary methods described in the present disclosure.

In some embodiments, the storage device 130 may include a random-access memory (RAM), a read-only memory (ROM), a mass storage, a removable storage, a volatile read-write memory, or any combination thereof. In some embodiments, the storage device 130 may be implemented on a cloud platform. In some embodiments, the storage device 130 may be connected to the network 140 to communicate with one or more components of the application scenario 100 (e.g., the imaging device 110, the processor 120, etc.). One or more components of the application scenario 100 may access data or instructions stored in the storage device 130 via the network 140. In some embodiments, the storage device 130 may be part of the processor 120.

The network 140 may include any suitable network that may facilitate the exchange of information and/or data in the application scenario 100. In some embodiments, one or more components of the application scenario 100 (e.g., the imaging device 110, the processor 120, or the storage device 130) may connect and/or communicate with other components of the application scenario 100 via the network 140.

In some embodiments, the network 140 may be any one or more of a wired network or a wireless network. For example, the network 140 may include a cable network, an optical fiber network, a telecommunications network, the Internet, local area networks (LANs), wide area networks (WANs), wireless local area networks (WLANs), metropolitan area networks (MANs), public switched telephone networks (PSTNs), Bluetooth networks, ZigBee networks, near-field communication (NFC), intra-device buses, intra-device wiring, cable connections, or any combination thereof. The network connections between components may be achieved using one of the aforementioned methods or a combination of a plurality of methods. In some embodiments, the network 140 may include one or more network access points. For example, the network 140 may include wired or wireless network access points, through which one or more components of the application scenario 100 may connect to the network 140 to exchange data and/or information.

In some embodiments, the application scenario 100 may also include a terminal device (not shown in FIG. 1).

The terminal device refers to one or more terminal devices or software used by a user. In some embodiments, the processor 120 may send a processing result to the terminal device to display the processing result to the user. For example, the processor 120 may send at least one of the medical image, the at least one property parameter of the vascular structure, and the hemodynamic characteristic of the vascular structure of the subject to the terminal device.

In some embodiments, the terminal device may be one or a combination of mobile devices, tablets, laptops, desktop computers, and other devices with input and/or output functions. In some embodiments, a user terminal may be used by one or more users, including users who directly use the services and other related users. The above examples are merely provided to illustrate the broad scope of the terminal devices and not to limit their scope.

It should be noted that the description of the above application scenario 100 is provided for illustrative purposes only and is not intended to limit the scope of the present disclosure. Various changes and modifications may be made by those skilled in the art under the teachings of the present disclosure.

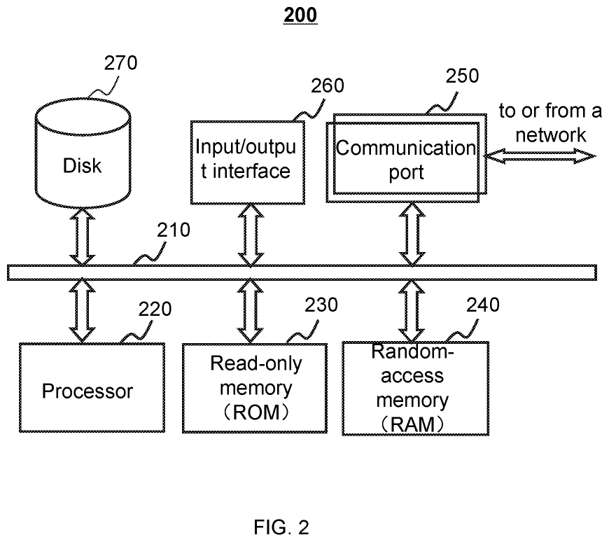

FIG. 2 is a schematic diagram illustrating hardware and software components of an exemplary computing device according to some embodiments of the present disclosure. The computing device 200 may be configured to perform one or more functions of at least one component in the system for evaluating a hemodynamic characteristic disclosed in the embodiments of the present disclosure.

The computing device 200 may be a general-purpose computer or a dedicated computer, both of which may be configured to implement the method for evaluating a hemodynamic characteristic described in some embodiments of the present disclosure. The computing device 200 may include any components to implement the system for evaluating a hemodynamic characteristic as described in the present disclosure. For example, the processor may be implemented on the computing device 200 through its hardware, software programs, firmware, or a combination of these. For convenience, only one computer is shown in the figure, but the computer functions related to the optimization of medical images described in the present disclosure may be implemented in a distributed manner on a plurality of similar platforms to distribute processing loads.

For example, the computing device 200 may include a communication port 250 connected to and/or from a network to enable data communication. The computing device 200 may also include a processor 220 in the form of one or more processors for executing program instructions. Exemplary computer platforms may include an internal communication bus 210, different types of program memory and data memory (such as a disk 270, a read-only memory (ROM) 230, or a random-access memory (RAM) 240), and various data files processed and/or transmitted by the computer. Exemplary computer platforms also include program instructions executed by the processor 220, which are stored in the ROM 230, RAM 240, and/or other forms of non-volatile storage media. The methods and/or processes of the present disclosure may be implemented in the form of program instructions. The computing device 200 may also include an input/output (I/O) interface 260 that supports input/output between the computer and other components. The computing device 200 may also receive programming and data through network communications.

For illustrative purposes only, only one CPU and/or processor in the computing device 200 is exemplarily described. However, it should be noted that the computing device 200 in some embodiments of the present disclosure may include a plurality of CPUs and/or processors. Therefore, the operations and/or methods implemented by one CPU and/or processor described in the present disclosure may also be implemented jointly or independently by a plurality of CPUs and/or processors. For example, if in the present disclosure, the CPU and/or processor of the computing device 200 performs operation A and operation B, it should be understood that operation A and operation B may also be performed jointly or independently by two different CPUs and/or processors in the computing device 200 (e.g., a first processor performs operation A, a second processor performs operation B, or the first and second processors jointly perform both operation A and operation B).

Those skilled in the art can understand that the structure shown in FIG. 2A is merely a block diagram of some structures related to the solutions described in the present disclosure and does not constitute a limitation on the computer devices to which the solutions are applied. Specific computer devices may include more or fewer components than those shown in the figure, or may combine certain components, or may have different component arrangements.

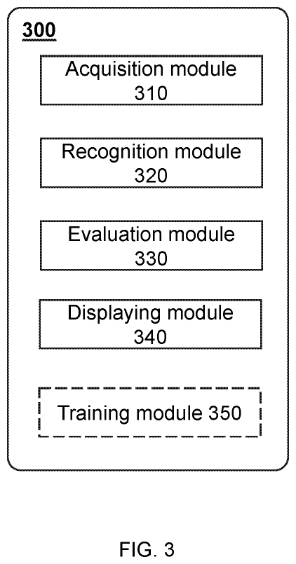

FIG. 3 is a block diagram illustrating an exemplary system for evaluating a hemodynamic characteristic according to some embodiments of the present disclosure. The system 300 for evaluating a hemodynamic characteristic may be implemented on the processor 120 in hardware and/or software form.

In some embodiments, as shown in FIG. 3, the system 300 for evaluating a hemodynamic characteristic may include an acquisition module 310, a recognition module 320, an evaluation module 330, and a displaying module 340.

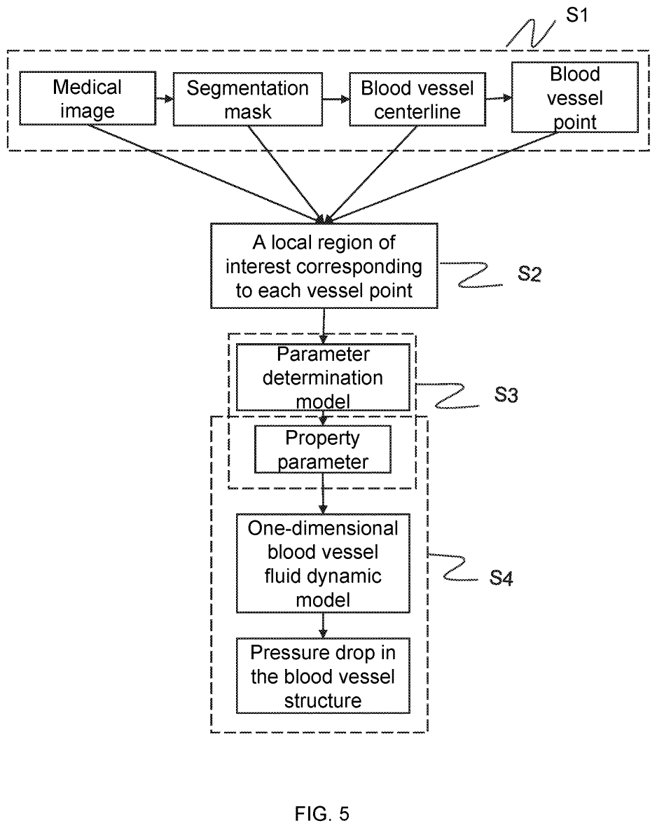

In some embodiments, the acquisition module 310 is configured to obtain a medical image of a subject, where the medical image includes at least a portion of a vascular structure. More information, please refer to FIG. 4 (e.g., operation 410) and FIG. 5 and its related descriptions.

In some embodiments, the recognition module 320 is configured to input the medical image into a trained parameter determination model to obtain at least one property parameter of the vascular structure. More information, please refer to FIG. 4 (e.g., operation 420) and FIG. 5 and its related descriptions.

In some embodiments, the evaluation module 330 is configured to input the at least one property parameter into a vascular fluid dynamic model for hemodynamic characteristic evaluation, resulting in at least one hemodynamic characteristic of the vascular structure. More information, please refer to FIG. 4 (e.g., operation 430) and FIG. 5 and theirs related descriptions.

In some embodiments, the displaying module 340 is configured to display the at least one hemodynamic characteristic of the vascular structure on a display device. More information, please refer to FIG. 4 (e.g., step 440) and its related descriptions.

In some embodiments, as shown in FIG. 3, the system 300 for evaluating the at least one hemodynamic characteristic may also include a training module 350.

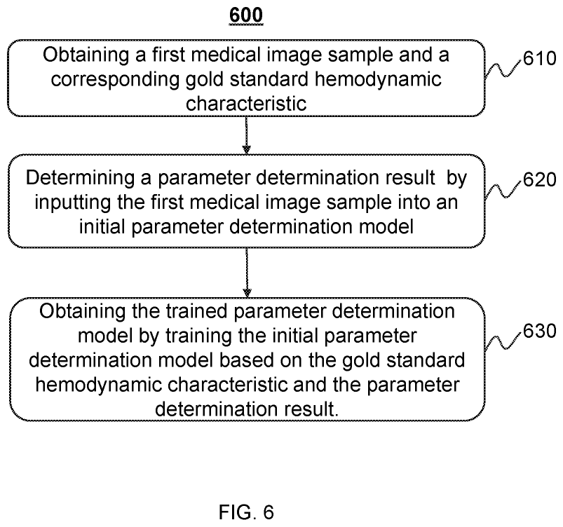

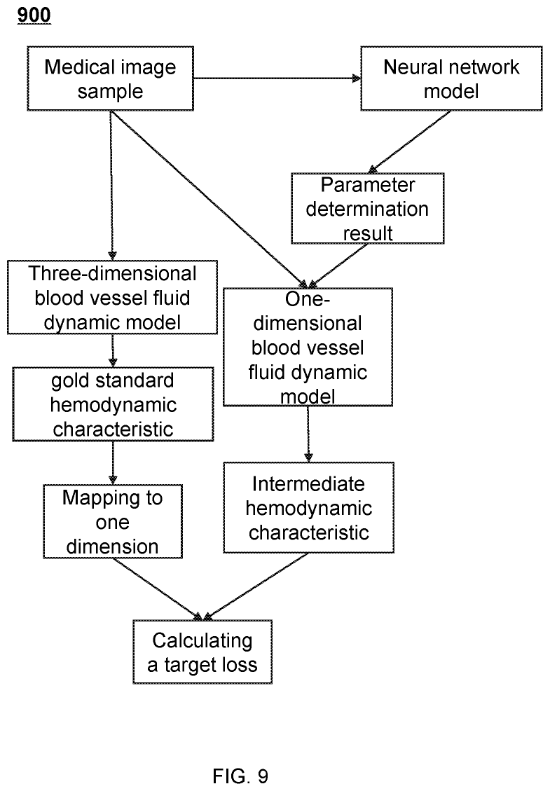

In some embodiments, the training module 350 is configured to obtain a plurality of first medical image samples and a plurality of corresponding gold standard hemodynamic characteristics; input the first medical image sample into an initial parameter determination model to obtain a parameter determination result of the initial parameter determination model; and train the initial parameter determination model based on the gold standard hemodynamic characteristic and the parameter determination result to obtain a trained parameter determination model. More information about the embodiment, please refer to FIG. 6 and FIG. 9 and their related descriptions.

In some embodiments, the training module 350 is also configured to input the parameter determination result into the vascular fluid dynamic model to obtain an intermediate hemodynamic characteristic; construct a target loss based on the gold standard hemodynamic characteristic and the intermediate hemodynamic characteristic; and adjust a parameter of the initial parameter determination model based on the target loss until the target loss function meets a termination condition, resulting in a trained parameter determination model. More information about the embodiment, please refer to FIG. 6 and FIG. 9 and their related descriptions.

In some embodiments, the intermediate hemodynamic characteristic and the gold standard hemodynamic characteristic respectively include at least two parameter types, and each parameter type corresponds to a weight. The training module 350 is also configured to determine a loss value corresponding to each parameter type based on the gold standard hemodynamic characteristic and the intermediate hemodynamic characteristic of the same parameter type; and determine the target loss based on the loss values and weights corresponding to each of the at least two parameter types. More information about the embodiment, please refer to FIG. 6 and FIG. 9 and their related descriptions.

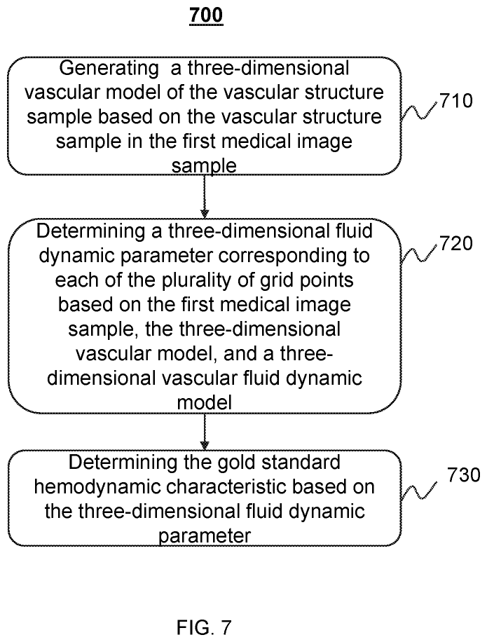

In some embodiments, the training module 350 is also configured to establish a three-dimensional (3D) vascular model of a vascular structure sample based on the vascular structure sample in the first medical image sample, wherein the 3D vascular model includes a plurality of mesh vertices. The training module 350 is further configured to determine a 3D fluid dynamic parameter corresponding to each mesh vertex based on the first medical image sample, the 3D vascular model, and a 3D vascular fluid dynamic model. The gold standard hemodynamic characteristic is then determined based on the 3D fluid dynamic parameter. For more information about the embodiment, please refer to FIG. 7 and FIG. 9 and their related descriptions.

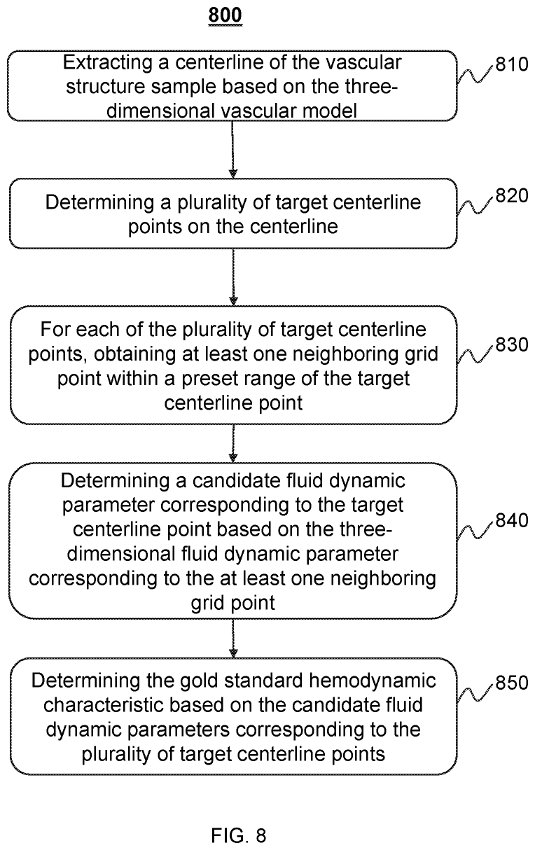

In some embodiments, the training module 350 is also configured to extract a centerline of the vascular structure sample based on the 3D vascular model. A plurality of target centerline points is identified on the centerline. For each target centerline point among the plurality of target centerline points, at least one neighboring mesh vertex within a preset range of the target centerline point is obtained. A candidate fluid dynamic parameter corresponding to the target centerline point is determined based on the 3D fluid dynamic parameter corresponding to the at least one neighboring mesh vertex. The gold standard hemodynamic characteristic is then determined based on the candidate fluid dynamic parameter corresponding to a plurality of target centerline points. For more information about the embodiment, please refer to FIG. 8 and FIG. 9 and their related descriptions.

In some embodiments, the training module 350 is also configured to treat the at least one property parameter of the vascular structure sample in the first medical image sample as the gold standard property parameter. Based on the first medical image sample and the corresponding gold standard property parameter, a preset neural network is trained to obtain an initial parameter determination model. For more information about the embodiment, please refer to FIG. 6 and FIG. 9 and their related descriptions.

In some embodiments, when the model training and/or updating process (e.g., processes 600-800) is executed by a processing device of a model provider, the training module 350 may be implemented by the processing device of the model provider. When the model training and/or updating process is executed by a processing device (e.g., the processor 120) of a model application end (e.g., a hospital), the training module 350 may be implemented by the processor 120. For example, the system 300 for evaluating a hemodynamic characteristic may include the training module 350 and be implemented by the processor 120. In some embodiments, when the model training process is executed by a processing device of a model provider, the system 300 for evaluating a hemodynamic characteristic implemented by the processor 120 may also include a training module, which may be configured to regularly update the trained parameter determination model.

It should be noted that the above descriptions of the system for evaluating a hemodynamic characteristic and its modules are for convenience only and do not limit the present disclosure to the embodiments provided. It can be understood that after understanding the principles of the system, those skilled in the art may combine various modules in any way without deviating from the principle, or form subsystems to connect with other modules. In some embodiments, the acquisition module 310, the recognition module 320, the evaluation module 330, and the training module 350 disclosed in FIG. 1 may be different modules in a system, or a single module may perform the functions of two or more of the above modules. For example, each module may share a common storage module, or each module may have its own storage module. Such variations are within the scope of protection of the present disclosure.

FIG. 4 is a flowchart illustrating an exemplary process for evaluating at least one hemodynamic characteristic according to some embodiments of the present disclosure. In some embodiments, the process 400 for evaluating at least one hemodynamic characteristic may be executed by a processor (e.g., the processor 120). For example, process 400 may be stored in a storage device (e.g., the storage device 130) in the form of a program or instructions, and when the processor executes the program or instructions, process 400 may be achieved. The schematic diagram of the operations of process 400 presented below is illustrative. In some embodiments, the process may be accomplished with one or more additional operations not described and/or one or more operations not discussed. Additionally, the order of the operations of process 400 shown in FIG. 4 and described below is not restrictive. As shown in FIG. 4, process 400 includes the following operations.

In 410, the processor 120 (e.g., the acquisition module 310) may obtain a medical image of a subject.

The subject refers to the subject that is to be scanned. In some embodiments, the subject may include a biological subject and/or a non-biological subject. For example, the subject may include a patient, an animal, an artificial subject, etc. In some embodiments, the subject may include a specific part of the body, such as the chest, etc. In some embodiments, the subject may include a specific organ, such as the heart, the esophagus, the trachea, the bronchi, a blood vessel, etc., or any combination thereof.

In some embodiments, the medical image may include the subject.

In some embodiments, the medical image may include a vascular tissue of the subject. The vascular tissue may include at least one of an arterial vessel, a coronary vessel, and a venous vessel.

In some embodiments, the medical image may include a vascular structure of the subject. The vascular structure may be at least one segment of a vascular, or the vascular structure may be the vascular structure at a specific position or point. For more information about the vascular point, please refer to operation 420 and its related descriptions.

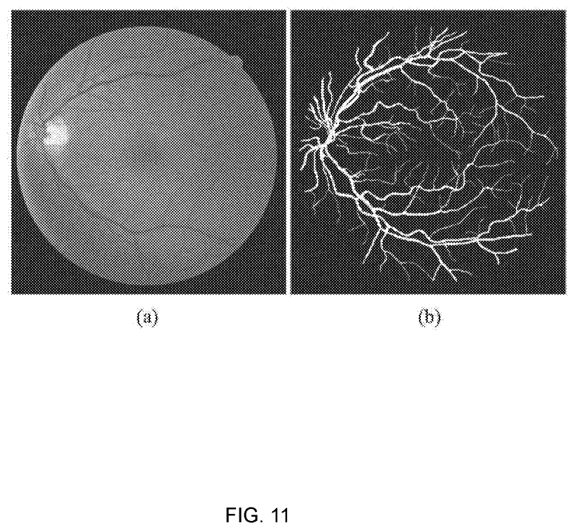

In some embodiments, the medical image may be a raw image. For example, the medical image may be an image directly acquired by a medical imaging device (e.g., the imaging device 110). In some embodiments, the medical image may be a segmented image. For example, the medical image may be an image obtained by segmenting at least a portion of the vascular structure from the original image. By way of example only, as shown in FIG. 11, image (a) in FIG. 11 is the original fundus image, and image (b) in FIG. 11 is the segmented image corresponding to the vascular structure segmented from image (a).

In some embodiments, the medical image may be a 2D image or a 3D image. In some embodiments, the medical image may be a static image or a dynamic image. For example, the dynamic image may include a sequence of 2D images or 3D images in chronological order.

In some embodiments, the medical image may be an image obtained through post-processing based on scanning data and/or original images acquired by the medical imaging device. For example, the medical image may be a multi-planar reconstructed image, a curved planar reconstructed image, a 3D rendered image, etc.

In some embodiments, the medical image may be a single-modal image or a multi-modal image. In some embodiments, the single-modal image may include various forms such as a computed tomography (CT) image, a positron emission computed tomography (PET) image, a single-photon emission computed tomography (SPECT) image, a magnetic resonance (MR) image, a digital subtraction angiography (DSA) image, and a computed tomography angiography (CT Angiography) image. In some embodiments, the multi-modal image may include various types such as a MR-CT image, a PET-MR image, a PET-CT image, a SPECT-CT image, and a SPECT-MR image. The embodiments of the present disclosure do not specifically limit the type of medical image.

In some embodiments, the processor may obtain the medical image of the subject from the medical imaging device, or from a picture archiving and communication system (PACS). In some embodiments, the processor may obtain the medical image of the subject from other storage devices or storage regions. In some embodiments, the processor may receive the medical image of the subject sent by other devices. The embodiments of the present disclosure do not specifically limit the method of obtaining the medical image.

In 420, the processor 120 (e.g., the recognition module 320) may determine at least one property parameter of the vascular structure by inputting the medical image into a trained parameter determination model.

The parameter determination model refers to a machine learning model configured to determine the at least one property parameter of the vascular structure. In some embodiments, the parameter determination model may be trained based on at least one neural network model such as a convolutional neural network (CNN), a fully connected network (FCN), or a self-attention network. For example, the parameter determination model may consist of the CNN and the FCN, or the parameter determination model may be composed of the self-attention Network model based on the transformer network architecture.

In some embodiments, the parameter determination model may involve at least 800 multiplication operations in a single execution. In some embodiments, at least part of the parameter determination model is executed by the GPU of the processor.

In some embodiments, an input of the parameter determination model may include the medical image. For example, the input of the parameter determination model may include one or more segmented images obtained by segmenting the vascular structure from the raw image. Alternatively, the input of the parameter determination model may include both the raw image and the segmented image derived from the raw image.

In some embodiments, the input of the parameter determination model may include one or more vascular points on the vascular structure in the medical image. For example, the processor may identify one or more vascular points on the vascular structure from the medical image and input the medical image with the annotated vascular points into the parameter determination model.

In some embodiments, a vascular point may correspond to a cross-section of the vascular structure. The cross-section of the vascular structure refers to a section corresponding to the normal direction at a point on the vascular centerline. In some embodiments, the processor may determine a bifurcation point in the vascular structure as a vascular point. In some embodiments, the processor may identify an endpoint of the vascular structure as a vascular point. In some embodiments, the processor may determine a vascular point at a preset distance along the vascular structure. The preset distance may be a system default, an empirical value, a predefined value, or any combination thereof, and may be set according to actual needs.

In other embodiments, the processor may determine an abnormal region within the vascular structure. For example, the processor may determine a region of vascular stenosis as the abnormal region. A vascular point may be then determined based on the abnormal region. For example, a point at the preset distance from of the abnormal region along the upstream and/or the downstream direction of the vascular structure may be determined as a vascular point. Alternatively, a midpoint of the abnormal region, or the narrowest point within the abnormal region may be designated as a vascular point.

In some embodiments, the processor may utilize any feasible image recognition algorithm and/or model to determine vascular point from the vascular structure in the medical image. In some embodiments, the vascular point may be manually annotated and determined in the medical image by relevant technicians (such as doctors, technicians, engineers, or nurses).

In some embodiments, the input of the parameter determination model may include an image of a local region or volume of interest corresponding to one or more vascular points in the vascular structure. In some embodiments, for each vascular point, the processor may extract a corresponding local volume of interest (VOI) or region of interest (ROI) image from the medical image and input the images of local regions or volumes of interest corresponding to one or more vascular points into the parameter determination model for property parameter recognition. The image of the local region or volume of interest corresponding to a vascular point may refer to an image of a region within the preset distance of the vascular point along the upstream and/or downstream direction in the vascular structure of the medical image.

In some embodiments, the processor may input the medical image (e.g., the original image and/or segmented image) into the parameter determination model, and the parameter determination model may perform a vascular point recognition operation to determine one or more vascular points. In some embodiments, the parameter determination model may further determine the image of the local region or volume of interest in the medical image corresponding to the one or more vascular points.

As an example, when extracting one or more vascular points from the vascular structure in the medical image, the processor may determine the vascular centerline corresponding to the vascular structure from the medical image. The processor may determine one or more vascular points on the vascular centerline. To determine the vascular centerline, the processor may input the medical image into a vascular segmentation model for vascular segmentation, and obtain a vascular segmentation mask. Subsequently, by processing the vascular segmentation mask, the processor may determine the vascular centerline corresponding to the vascular structure. Of course, the processor may adopt other image processing methods to obtain the vascular centerline corresponding to the vascular structure from the medical image.

An output of the parameter determination model may include the at least one property parameter of the vascular structure contained in the medical image. The at least one property parameter may include property parameters corresponding to one or more vascular points of the vascular structure.

In some embodiments, for a vascular point where the cross-section is circular, a corresponding actual morphological parameter may include at least one of an actual diameter, an actual radius, an actual cross-sectional area, and an actual cross-sectional perimeter of the cross-section. However, when the cross-section corresponding to the vascular point is non-circular (e.g., due to deformation or vascular abnormalities such as stenosis, occlusion, aneurysm, vascular wall calcification, resulting in an irregular cross-section), it becomes challenging to obtain the actual morphological parameter of the cross-section.

In some embodiments, for a vascular point where the cross-section is non-circular the processor may determine at least one of an equivalent diameter, an equivalent radius, an equivalent cross-sectional area, and an equivalent cross-sectional perimeter of the vascular structure.

In some embodiments, the at least one property parameter corresponding to the vascular point may include at least one of the equivalent diameter, the equivalent radius, the equivalent cross-sectional area, and the equivalent cross-sectional perimeter of the cross-section corresponding to that vascular point.

For a vascular point where the corresponding cross-section is circular, the at least one property parameter output by the parameter determination model is closer to the actual morphological parameter of the cross-section. However, when the corresponding cross-section is non-circular, the at least one property parameter output by the parameter determination model do not represent the actual morphological parameter of the vascular structure, and there is no standard value for the property parameter. In the present disclosure, the determination of the property parameter is not solely based on vascular morphology but also considers the performance of vascular fluid dynamics. For more information about the determining the property parameter, please refer to the relevant descriptions in the present disclosure.

Relationships between the equivalent diameter, the equivalent radius, the equivalent cross-sectional area, and the equivalent cross-sectional perimeter may be determined based on formular for circular. For example, the equivalent diameter d is equal to twice the equivalent radius r. The equivalent cross-sectional area S is determined according to formula S=πr². The equivalent cross-sectional perimeter is determined according to formula L=πd.

In some embodiments, the input of the parameter determination model may be the raw image containing the vascular structure, and the output of the parameter determination model may be the property parameter of the vascular structure. Alternatively, the output of the parameter determination model may be the equivalent diameter of the vascular structure. For more information about the raw image, please refer to operation 410 and its related descriptions.

In some embodiments, the input of the parameter determination model may be a segmented image corresponding to the vascular structure, and the output of the parameter determination model may be the at least one property parameter of the vascular structure. Alternatively, the output of the parameter determination model may be the equivalent diameter of the vascular structure. For more information about the segmented images, please refer to operation 410 and its related descriptions.

In some embodiments, the input of the parameter determination model may include both the raw image and the segmented image corresponding to the vascular structure, and the output of the parameter determination model may be the at least one property parameter of the vascular structure. Alternatively, the output of the parameter determination model may specifically be the equivalent diameter of the vascular structure.

In other embodiments, the input of the parameter determination model may include at least one physiological parameter and/or basic information of the subject (e.g., the patient).

In some embodiments, the at least one physiological parameter of the subject may include at least one of a respiratory rate, a heart rate, a blood pressure (e.g., measured by a blood pressure monitor), a blood oxygen level, a blood glucose level, a complete blood count, etc., or a combination thereof.

In some embodiments, the basic information of the subject may include a gender, an age, a height, a weight, a medical history, lifestyle habits (such as a smoking history) of the subject, or the like, or a combination thereof.

In some embodiments, the processor may determine the at least one physiological parameter and/or the basic information of the subject by acquiring user input (e.g., from a doctor, a nurse, or a patient). Alternatively, the processor may retrieve the at least one physiological parameter and/or the basic information the from other storage devices or regions. In some embodiments, the processor may receive the at least one physiological parameter and/or the basic information from other devices. The specific method of obtaining the at least one physiological parameter and/or the basic information is not limited in the present disclosure.

In some embodiments, the parameter determination model may output one or more property parameters. For example, the parameter determination model may output at least one of the equivalent diameter, the equivalent radius, the equivalent cross-sectional area, and the equivalent cross-sectional perimeter of the cross-section corresponding to that vascular point. In some embodiments, when the parameter determination model is required to output a plurality of property parameters at one time, the parameter determination model may be set to include a plurality output nodes each of which is configured to output one of the plurality of property parameters. For example, the parameter determination model may include two output nodes each of which is configured to output the equivalent diameter and the equivalent cross-sectional area, respectively. In other embodiments, when the parameter determination model is required to output a plurality of property parameters at one time, a plurality of processing layers (i.e., parameter determination sub-models) may be set up in the parameter determination model, and different parameter determination sub-models are used to output different property parameters. For example, a parameter determination sub-model may be configured to output the equivalent diameter, and another parameter determination sub-model may be configured to output the equivalent cross-sectional area.

In some embodiments, the at least one property parameter includes parameters of at least two types, the parameter determination model includes at least two nodes or at least two parameter determination sub-models each of which is configured to output the parameters of at least one type. For example, the at least two types of property parameter may include at least two of the equivalent diameter, the equivalent radius, the equivalent cross-sectional area, and the equivalent cross-sectional perimeter of the cross-section corresponding to that vascular point.

In some embodiments, the parameter determination model may be obtained through training. The processor may obtain an initial parameter determination model and train the initial parameter determination model to obtain the trained parameter determination model. For more information about the initial parameter determination model and the training process of the initial parameter determination model, please refer to FIG. 6 and its related descriptions.

In 430, the processor 120 (e.g., the recognition module 320) may determine at least one hemodynamic characteristic of the vascular structure by inputting the at least one property parameter into a vascular fluid dynamic model for a hemodynamic characteristic evaluation. The hemodynamic characteristic of the vascular structure may include one or more hemodynamic characteristics corresponding to one or more vascular points within the vascular structure. A hemodynamic characteristic of a vascular point refers to a hemodynamic characteristic determined based on the at least one property parameter specific to that point.

The vascular fluid dynamic model may be a vascular evaluation model grounded in computational fluid dynamics (CFD). The vascular fluid dynamic model may be used to evaluate the hemodynamic characteristic of the vascular structure.

The hemodynamic characteristic is data reflecting features of blood flow in blood vessels. In some embodiments, the hemodynamic characteristic refers to a parameter related to a blood flow in a one-dimensional space. In some embodiments, the at least one hemodynamic characteristic may include at least one type from a blood pressure, a pressure drop, a flow velocity, a wall shear stress, a circumferential stress, a fractional flow reserve (FFR), a blood flow volume, a blood viscosity, or a blood flow morphology of the vascular structure. The FFR is useful for evaluating a coronary artery lesion and impact of the coronary artery lesion on downstream blood supply.

In some embodiments, the at least one hemodynamic characteristic may include characteristics of at least two types. For example, the at least two types of hemodynamic characteristic may include at least two of a blood pressure, a flow velocity, a wall shear stress, a circumferential stress, a fractional flow reserve (FFR), a blood flow volume, a blood viscosity, and a blood flow morphology of the vascular structure. The at least one property parameter output by each of the at least two parameter determination sub-models is configured to determine the characteristics of at least one type.

In scenarios where the at least one hemodynamic characteristic includes at least two types, the vascular fluid dynamic model may include at least two parameter determination sub-models for evaluating different types of at least one hemodynamic characteristic. The sub-models may include a sub-model for evaluating a blood pressure, a sub-model for evaluating a flow velocity, etc. In some embodiments, different sub-models may require the same or different property parameters of the vascular structure for their respective evaluations.

In embodiments where the vascular fluid dynamic model includes one or more of sub-models, the processor may determine the one or more property parameters needed for each sub-model and input the one or more property parameters for corresponding hemodynamic characteristic evaluations into a corresponding sub-model to obtain a specific evaluation result of each sub-model.

It should be noted that when the at least one property parameter of the vascular structure include property parameters from a plurality of vascular points, the processor may identify the property parameters corresponding to different sub-models for each point. Then, for each sub-model, a relevant property parameter may be inputted into the sub-model to evaluate a corresponding hemodynamic characteristic, resulting in a specific evaluation result of each vascular point.

For example, in the blood pressure evaluation, if the sub-model requires the vessel diameter at a specific vascular point, the processor may determine the vessel diameter from the at least one property parameter and input the vessel parameter into the sub-model for evaluation, thus obtaining a blood pressure value at that the specific vascular point.

Taking the pressure drop as an example, the at least one property parameter may be inputted into a one-dimensional vascular fluid dynamic model for blood pressure evaluation, the pressure drop within the vascular structure may be determined. For more information about the one-dimensional vascular fluid dynamic model for blood pressure evaluation, please refer to FIG. 5 and its related description.

In some embodiments, the vascular fluid dynamic model includes a three-dimensional vascular fluid dynamic model.

In certain embodiments, the vascular fluid dynamic model includes a reduced-order model and/or a reduced-dimensionality model of a three-dimensional vascular fluid dynamic model.

The three-dimensional vascular fluid dynamic model is a CFD-based vascular evaluation model where the vascular structure is represented in three dimensions. The hemodynamic characteristic determined using the three-dimensional vascular fluid dynamic I model may generate a three-dimensional CFD result.

The three-dimensional vascular fluid dynamic model may include mathematical models include a differential equation, a difference equation, or the like.

The reduced-order model of the three-dimensional vascular fluid dynamic model may be obtained by neglecting higher-order terms, resulting in a lower-order model. Model order-reduction is a technique to simplify the model and reduce the model computation under the premise of guaranteeing the accuracy.

The reduced-dimensionality model of the three-dimensional vascular fluid dynamic model may be a low-dimensional model that transforms the data (e.g., vascular structure) into a low-dimensional representation. In the reduced-dimensionality model of the three-dimensional vascular fluid dynamic model, the vascular structure may be represented in a reduced dimension. For example, the vascular structure may be simplified to be denoted by a one-dimensional form, where the vascular structure is denoted by the centerline of the vascular structure.

In some embodiments, the vascular fluid dynamic model may be a one-dimensional vascular fluid dynamic model, derived from the three-dimensional vascular fluid dynamic model after reducing the dimensionality and order. That is, the one-dimensional vascular fluid dynamic mode may be a lower-order low-dimensional model that both neglects higher-order terms and transforms the data (e.g., vascular structure) into a low-dimensional representation.

When using the three-dimensional vascular fluid dynamic model to determine the hemodynamic characteristic a three-dimensional vascular model may be constructed, the three-dimensional vascular model may be meshed, boundary conditions like pressure and flow rate may be determined, and discretized differential equations may be solved based on the boundary conditions to obtain blood flow information such as blood pressure and flow velocity in three dimensions. Due to the complexity of constructing a three-dimensional vascular model (considering structures like plaques) and the computational intensity of solving the three-dimensional vascular model, some embodiments of the present disclosure adopt the reduced-order and/or the reduced-dimensionality models to simplify the solution process and reduce computation time.

To address challenges associated with using the one-dimensional vascular fluid dynamics model, some embodiments of the present disclosure may determine at least one of the equivalent diameters, the equivalent radius, the equivalent cross-sectional area, or the equivalent cross-sectional perimeter of the vascular structure. These equivalent parameters are then inputted into the one-dimensional model for determining hemodynamic characteristic, simplifying the process and improving computational speed.

In some embodiments, the processor 120 (e.g., the displaying module 340) may display the at least one hemodynamic characteristic of the vascular structure on a display device.

The forms of displaying the at least one hemodynamic characteristic of the vascular structure on the display device include a variety of forms. In some embodiments, the displaying module 340 may use text, voice, images, video, etc., to display the at least one hemodynamic characteristic of the vascular structure on the display device. For example, the displaying module 340 may display the at least one hemodynamic characteristic of the vascular structure in the form of a table or a report. As another example, the displaying module 340 may label the at least one hemodynamic characteristic of the vascular structure on a 3D vascular model of the vascular structure or a medical image.

In some embodiments, the displaying module 340 may display the at least one property parameter of the vascular structure and/or the medical image of the subject on the display device. In some embodiments, the displaying module 340 may display the one or more vascular points on the vascular structure in the medical image. For more information about the vascular point, please refer to operation 420 and its related descriptions.

In some embodiments, the displaying module 340 may display an image of a local region or volume of interest corresponding to one or more vascular points in the vascular structure in the medical image.

In some embodiments, the displaying module 340 may display at least one physiological parameter and/or basic information of the subject on the display device.

When using the reduced-order model or the reduced-dimensionality model (e.g., the vascular fluid dynamics model) of the three-dimensional vascular fluid dynamics model to calculate the hemodynamic characteristic, it is important to note that the one-dimensional model cannot capture all the information contained in the three-dimensional model. Even when the vessel diameter parameters calculated from the three-dimensional model are applied to the one-dimensional model, the vessel diameter parameters still only contain partial information. For example, an eccentric plaque and a circumferential plaque may both cause vascular stenosis with the same diameter, but their hemodynamic performances are completely different, and their impacts on downstream blood pressure and flow are also distinct. Such structural differences are easily identifiable in the three-dimensional vascular fluid dynamics model, but due to their identical diameters, they appear identical in one-dimensional models, leading to inaccuracies in calculation results.

Some embodiments of the present disclosure may determine at least one of the equivalent diameter, equivalent radius, equivalent cross-sectional area, and equivalent cross-sectional perimeter of the vascular structure. These parameters may then be input into a one-dimensional vascular fluid dynamics model for hemodynamic characteristic calculation, simplifying the calculation process and improving computation speed. Additionally, by inputting at least one of the equivalent diameter, equivalent radius, equivalent cross-sectional area, and equivalent cross-sectional perimeter into the one-dimensional hemodynamic model, some embodiments of this specification can obtain hemodynamic characteristics that are closer to those evaluated based on complex three-dimensional vascular fluid dynamics models, which not only reduces the computational complexity of the three-dimensional vascular fluid dynamics model but also further improves the accuracy of hemodynamic characteristic evaluation.

FIG. 5 is a schematic diagram illustrating an exemplary process for evaluating a hemodynamic characteristic according to some embodiments of the present disclosure. As shown in FIG. 5, taking the hemodynamic characteristic including a pressure drop (a blood pressure drop) as an example, the evaluation of the hemodynamic characteristic may include the following operations:

In S1, the processor 120 (e.g., the acquisition module 310) may obtain a medical image of a subject, perform a vascular segmentation operation on the medical image to obtain a vascular segmentation mask, determine a vascular centerline based on the vascular segmentation mask, and identify a plurality of vascular points from the vascular centerline.

In S2, the processor 120 (e.g., the acquisition module 310) may extract a local region of interest corresponding to each of the plurality of vascular points from the medical image. More information about operations S1 and S2, please refer to operation 410 in FIG. 4 and its related descriptions.

In S3, the processor 120 (e.g., the recognition module 320) may input the local region of interest for each vascular point into a trained parameter determination model for recognition to obtain at least one property parameter of each vascular point. The at least one property parameter of a vascular point may include an equivalent diameter at the vascular point. For more information about operation S3, please refer to operation 420 in FIG. 4 and its related descriptions.

Optionally, for each vascular point, the local region of interest and its corresponding local vascular segmentation mask may be input into the trained parameter determination model for recognition to obtain the equivalent diameter of the vascular point.

In some embodiments, the processor may input at least one physiological parameter and/or basic information of the subject (e.g., the patient) into the trained parameter determination model. For more information about the physiological parameter and the basic information of the subject refer to FIG. 4 and its related descriptions.

In some embodiments, the processor may mark the local blood vessels in the local region of interest as a masked region, segment the masked region out of the local region of interest to obtain a local vascular segmentation mask representing the position and shape of the local blood vessels.

In S4, for each vascular point, the processor 120 (e.g., the evaluation module 330) may input the at least one property parameter of the vascular point into a one-dimensional vascular fluid dynamic model for blood pressure evaluation, to obtain the pressure drop of the vascular structure.

In some embodiments, the one-dimensional vascular fluid dynamic model for blood pressure evaluation may be represented by the following formula (1) or variations of formula (1):

$$Δ P = {c}_{1} V + {c}_{2} {V}^{2} , \qquad ( 1 )$$

where ΔP represents the pressure drop across a segment of a vascular, which may be a segment between an initial vascular point and a target vascular point. For example, the segment of the vascular may be an abnormal vascular segment that includes stenosis. V represents the average blood flow velocity in the vascular segment; c₁ is the viscosity loss coefficient, indicating the retardation effect of the wall friction on blood flow; c₂ is the expansion loss coefficient, indicating the retardation effect of the stenosis on blood flow. The initial vascular point refers to a starting endpoint of the vessel segment. The target vascular point refers to a tail endpoint of the vascular segment. The initial vascular point is located before the target vascular point, meaning that the blood in the vascular segment flows through the initial vascular point first and then through the target vascular point.

In some embodiments, the viscosity loss coefficient c₁ may be determined according to the following formula (2):

$${c}_{1} = 128 * μ * {L}_{tot} * \frac{{A}_{i n}}{π {D}^{4}} , \qquad ( 2 )$$

where μ represents blood viscosity, Lₜₒₜ represents the length of the vascular segment, Aᵢₙ represents the cross-sectional area at the start of the lumen (initial vascular point), and D represents the diameter of the lumen at a stenosed position (for example, the middle or the narrowest position of a stenosed region of the lumen).

It should be noted that blood viscosity μ, the length of the vascular segment Lₜₒₜ, and the cross-sectional area at the start of the lumen Aᵢₙ are all parameters independent of the degree of stenosis. Therefore, the blood viscosity μ may be a fixed value. The length of the vascular segment Lₜₒₜ may be arbitrarily selected by the user (e.g., a doctor or a nurse, etc.). Since there is no stenosis at the start of the lumen, it may be approximated as circular, and the processor may determine the cross-sectional area Aᵢₙ at the start of the lumen according to the formula for determining the area of a circle.

Due to the presence of stenosis, the lumen at the stenosed region is not circular. In a traditional method, the hydraulic diameter is used as the diameter D of the stenosed position of the lumen, which is determined as the ratio of the cross-sectional area A of the drainage basin at the stenosed position to the perimeter P of the vascular cross-section at the stenosed region. However, the hydraulic diameter not only fails to accurately represent the actual diameter at the stenosed position, but the determination of the cross-sectional area A of the drainage basin at the stenosed position is also relatively complex. Therefore, in some embodiments of the present disclosure, a parameter determination model may be configured to determine the equivalent diameter at the stenosed position. For more information about the parameter determination model, please refer to FIG. 4 and FIG. 6, and their related descriptions.

In some embodiments, the expansion loss coefficient c₂ may be determined using the following formula (3):

$${c}_{2} = ρ / ( 2 λ ) , \qquad ( 3 )$$

where ρ represents a blood density and λ is a first empirical parameter. In some embodiments, the processor may determine the first empirical parameter λ based on the at least one physiological parameter and/or the basic information of the subject. For example, when the subject has a higher age, greater weight, or a history of smoking, their vascular elasticity is lower, making vascular stenosis more likely to cause elevated blood pressure. In the case, it is necessary to reduce the first empirical parameter λ to increase the expansion loss coefficient c₂.

When the one-dimensional vascular fluid dynamic model is determined according to formula (2) and formula (3), the required property parameter of the one-dimensional vascular fluid dynamic model may include the diameter D of the stenosed lumen, which may be identified using a parameter determination model.

In other embodiments, the expansion loss coefficient c₂ may be determined using the following formula (4) or variations of formula (4):

$${c}_{2} = ( ρ / 2 ) * ( {K}_{e} / 1333 ) * {( {A}_{i n} / {A}_{s t e n} - 1 )}^{2} , \qquad ( 4 )$$

where ρ represents blood density, Kₑ is the second empirical parameter, Aᵢₙ represents the cross-sectional area at the beginning of the lumen, and Aₛₜₑₙ represents the cross-sectional area at the stenosed part of the lumen. In some embodiments, the processor may determine the second empirical parameter Kₑ based on the at least one physiological parameter and/or the basic information of the subject. The determination method of the second empirical parameter Kₑ is similar to that of the first empirical parameter A. The second empirical parameter Kₑ may be the same as the first empirical parameter A, or it may be different.

When the one-dimensional vascular fluid dynamic model is determined according to formula (2) and formula (4), the required property parameter of the one-dimensional vascular fluid dynamic model include the area at the stenosed part of the lumen, Aₛₜₑₙ, which is related to the diameter D at the stenosed part. Therefore, the parameter determination model may be configured to determine both the diameter D and the area Aₛₜₑₙ at the stenosed part of the lumen. Alternatively, the parameter determination model may be configured to determine one of the diameter D and the area Aₛₜₑₙ at the stenosed position, and then the other one of the diameter D and the area Aₛₜₑₙ may be determined using the formula for the area of a circle.

In some embodiments, the cross-sectional area Aᵢₙ at the start of the lumen may be determined using a parameter determination model. Since there is no stenosis at the start of the lumen, it may be approximated as circular, and the equivalent cross-sectional area output by the parameter determination model at the start of the lumen is closer to the actual cross-sectional area.

For example, a medical image I includes a vascular segment A, which contains a stenosed region B. A vascular point C is determined based on the stenosed region B. The parameter determination model outputs at least one property parameter corresponding to the vascular point C based on the medical image I. The at least one property parameter is then input into a one-dimensional vascular fluid dynamic model (for example, a model constructed based on formulas (1), (2), (3) or (1), (2), (4)) to determine a pressure drop in the vascular segment A. The pressure drop in the vascular segment A represents a hemodynamic characteristic corresponding to vascular point C.

As another example, the medical image I includes a vascular segment A with a stenosed region B. A vascular point C is determined based on the stenosed region B, and a starting point of the vascular segment A is identified as a vascular point P. The parameter determination model outputs property parameters corresponding to both the vascular point C and the vascular point P based on the medical image I. These property parameters corresponding to both the vascular point C and the vascular point P are input into a one-dimensional vascular fluid dynamic model (such as a model based on formulas (1), (2), (3) or (1), (2), (4)) to determine a pressure drop in the vascular segment A. The pressure drop represents hemodynamic characteristics corresponding to both the vascular point C and the vascular point P.

For the aforementioned embodiments, if a blood pressure value at the starting vascular point P is known, a blood pressure value at the tail end of the vascular segment A may be further determined based on the pressure drop in the vascular segment A and the blood pressure value at the starting vascular point P. Alternatively, if the blood pressure value at the tail end of vascular segment A is known, the blood pressure value at the starting vascular point P may be determined based on the pressure drop in the vascular segment A and the blood pressure value at the tail end.

In some embodiments, any segment that includes the stenosed region B may be determined from the vascular segment A. The blood pressure value at any point upstream and/or downstream of the stenosed region B in the vascular segment A may be determined using the aforementioned method. For example, vascular segments A1, A2, A3 including the stenosed region B may be selected from the vascular segment A. The pressure drops in vascular segments A1, A2, A3 are determined separately using the aforementioned method. These pressure drops in vascular segments A1, A2, A3 represent the hemodynamic characteristics corresponding to the vascular point C. If the blood pressure values at the starting points of vascular segments A1, A2, A3 are known, the blood pressure values at the tail ends of vascular segments A1, A2, A3 (i.e., the blood pressure values at three points downstream of the stenosed region B) may be further determined based on the pressure drops in vascular segments A1, A2, A3 and the known blood pressure values at the starting points of vascular segments A1, A2, A3.

In some embodiments of the present disclosure, a machine learning model is configured to perform equivalent determination on the vascular diameter at specific points. The determined equivalent diameters are then configured to replace traditional hydraulic diameters in the vascular fluid dynamic model to evaluate the blood pressure at those points. Since the machine learning model may learn to determine equivalent diameters that are closer to the actual diameters, and because some embodiments of the present disclosure are trained using gold standard hemodynamic characteristics, the accuracy of the equivalent diameters may be further improved. Therefore, using the equivalent diameters determined by some embodiments of the present disclosure may address the limitations of hydraulic diameters, providing a more advantageous evaluation of fluid dynamics.

FIG. 6 is a flowchart illustrating an exemplary process for training a parameter determination model according to some embodiments of the present disclosure. In some embodiments, the process of training the parameter determination model may be executed by a processor (e.g., the processor 120 or other processing devices). For example, process 600 may be stored in a storage device (e.g., the storage device 130) in the form of a program or instructions, and when the processor executes the program or instructions, process 600 may be implemented. The operational schematic diagram of process 600 presented below is illustrative. In some embodiments, the process may be completed using one or more additional operations not described and/or one or more operations not discussed. Additionally, the order of the operations of process 600 shown in FIG. 6 and described below is not restrictive. As shown in FIG. 6, process 600 includes the following operations.

In 610, the processor 120 (for example, the training module 350) may obtain a first medical image sample and a corresponding gold standard of a hemodynamic characteristic (i.e., a first training label).

The first training sample may include a first medical image sample.

In some embodiments, the first medical image sample may include a vascular structure sample of a sample subject. The sample subject may be similar to the subject. The first medical image sample may be similar to the medical image of the subject. The vascular structure sample in the first medical image sample may be similar to f the vascular structure in the medical image of the subject.

In some embodiments, the processor may obtain the first medical image sample based on historical data. In some embodiments, the processor may obtain the first medical image sample based on simulated experiments.

In some embodiments, one or more vascular point samples may be determined in the first medical image sample. The first medical image sample annotated with the one or more vascular point samples may be used as the first training sample for model training. The determination of vascular point samples may be performed similar as the determination of vascular points as described in FIG. 4.

The gold standard of the hemodynamic characteristic is a training label configured to train the parameter determination model.

In some embodiments, the gold standard of the hemodynamic characteristic may be a hemodynamic characteristic determined based on a three-dimensional vascular fluid dynamic model.

In some embodiments, the gold standard of the hemodynamic characteristic may include a value of the hemodynamic characteristic of the vascular structure sample in the first medical image sample. In some embodiments, the gold standard of the hemodynamic characteristic may include the gold standard of the hemodynamic characteristics corresponding to one or more vascular point samples on the vascular structure sample in the first medical image sample.

In some embodiments, the processor may obtain the hemodynamic characteristics of each vascular point sample in the first medical image sample based on the blood flow evaluation of the three-dimensional vascular fluid dynamic model, which are used as the gold standard of the hemodynamic characteristics.

When determining the gold standard of the hemodynamic characteristic based on the three-dimensional vascular fluid dynamic model, the processor may establish a three-dimensional vascular model of the vascular structure sample based on the vascular structure sample in the first medical image sample, where the three-dimensional vascular model includes a plurality of mesh vertices. Based on the first medical image sample, the three-dimensional vascular model, and the three-dimensional vascular fluid dynamic model, the processor may determine a three-dimensional fluid dynamic parameter corresponding to each mesh vertex in the three-dimensional vascular model. The gold standard of the hemodynamic characteristic may be then determined based on the three-dimensional fluid dynamic parameter. For more information about the embodiment, please refer to FIG. 9 and its related description.

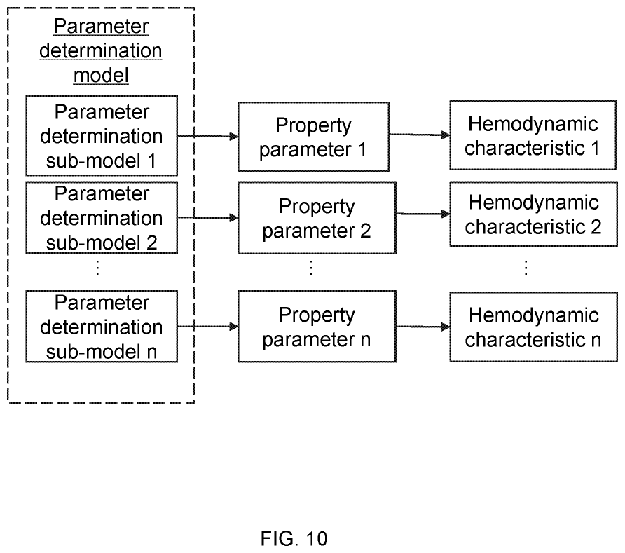

When the vascular fluid dynamic model is a reduced-order and/or reduced-dimensionality model of the three-dimensional vascular fluid dynamic model (for example, a reduced one-dimensional vascular fluid dynamic model after order and dimension reduction), the processor may further map a three-dimensional CFD result obtained based on the three-dimensional vascular fluid dynamic model to one dimension to obtain the hemodynamic characteristic under the one-dimensional vascular fluid dynamic model. The one-dimensional hemodynamic characteristic may be then used as the gold standard of the hemodynamic characteristic for training the parameter determination model. Therefore, in some embodiments, the processor may extract a centerline of the vascular structure sample based on the three-dimensional vascular model, determine a plurality of target centerline points on the centerline, and for each target centerline point among the plurality of target centerline points, obtain at least one neighboring mesh vertex within a preset range of the target centerline point. Based on a three-dimensional fluid dynamic parameter corresponding to the at least one neighboring mesh vertex, the processor may determine a candidate fluid dynamic parameter corresponding to the target centerline point. The gold standard of the hemodynamic characteristic may be determined based on the candidate fluid dynamic parameters corresponding to the plurality of target centerline points. For more information about the embodiment, please refer to FIG. 10 and its related description.

In 620, the processor 120 (for example, the training module 350) may determine a determination result from the initial parameter determination model by inputting the first medical image sample into the initial parameter determination model.

In some embodiments, the initial parameter determination model may be a machine learning model that has not undergone any training. In some embodiments, after constructing the basic model structure, the processor may obtain the initial parameter determination model through parameter initialization. In some embodiments, the processor may initialize the parameters by assigning zero values, unity values, or random values to the model parameters. In some embodiments, the processor may assign random values to the model parameters through random initialization methods such as Gaussian distribution initialization or uniform distribution initialization. In some embodiments, the initial parameter determination model may be a pre-trained machine learning model. For more information about pre-training will be described later.

In some embodiments, the determination result of the initial parameter determination model may include the property parameter of the vascular structure sample contained in the first medical image sample. For example, the processor may input the first medical image sample into the initial parameter determination model to obtain the property parameter of the vascular structure sample contained in the first medical image sample.

In some embodiments, the parameter determination result of the initial parameter determination model may include property parameters corresponding to one or more vascular point samples of the vascular structure sample contained in the first medical image sample. For example, the parameter determination result of the initial parameter determination model may include property parameters corresponding to various vascular point samples on the centerline of the blood vessels in the first medical image sample. For instance, the processor may obtain local region of interest images corresponding to various vascular point samples on the centerline of the blood vessels from the first medical image sample, and for each vascular point sample, input the local region of interest image corresponding to the vascular point sample into the initial parameter determination model for determination to obtain the property parameter corresponding to the vascular point sample. Similarly, the processor may obtain property parameters corresponding to various vascular point samples on the centerline of the blood vessels in the way, that is, the determination result may include property parameters corresponding to various vascular point samples on the centerline of the blood vessels in the first medical image sample.

To improve the accuracy of the vascular fluid dynamic model (for example, a reduced-order and/or reduced-dimensionality model of a three-dimensional vascular fluid dynamic model), it is necessary to ensure the accuracy of the property parameters output by the parameter determination model. Therefore, the output precision of the parameter determination model is crucial. During the training of the parameter determination model, it is relatively easy to obtain property parameters of normal vascular structures, but it is more difficult to determine property parameters of abnormal vascular structures such as lesions or stenoses. However, the parameter determination model needs to accurately identify property parameters for abnormal vascular structures. If theoretical property parameters of the vascular structure (for example, true morphological parameters) are used as the gold standard for model training to iteratively train the parameter determination model, it may be difficult to implement, and the trained parameter determination model may not perform well in recognizing property parameters of abnormal vascular structures.

Therefore, some embodiments of the present disclosure propose a scheme for guiding the training of the parameter determination model based on the hemodynamic characteristic, that is, based on the hemodynamic characteristic. In other words, during the training process of the initial parameter determination model, loss values may be determined based on the property parameters output by the parameter determination model, the hemodynamic characteristics determined by the vascular fluid dynamic model (for example, a reduced-order and/or reduced-dimensionality model of a three-dimensional vascular fluid dynamic model), and the corresponding gold standard of the hemodynamic characteristics. The model parameters of the parameter determination model may be then updated accordingly to complete the model training and obtain a well-trained parameter determination model. For more information on the embodiment, please refer to operation 630 and its related description.

In some embodiments, the first training sample may further include at least one of a physiological parameter sample of the sample subject, basic information sample of the sample subject, and a local region of interest image corresponding to the vascular point sample.

In 630, the processor 120 (for example, the training module 350) may obtain a trained parameter determination model by training the initial parameter determination model based on the gold standard hemodynamic characteristic and the parameter determination result.

In some embodiments, the processor may train the initial parameter determination model based on a plurality of first training samples with first labels using various methods, to update the parameters of the initial parameter determination model to obtain the trained parameter determination model. For example, training of the initial parameter determination model may be performed based on the gradient descent algorithm. In some embodiments, the first labels may include gold standard of the hemodynamic characteristics corresponding to the first training samples.

In some embodiments, during the process of updating the parameters of the initial parameter determination model, the processor may select an appropriate optimizer and loss function, and set an appropriate training plan to update the parameters of the initial parameter determination model.

In some embodiments, during the process of updating the parameters of the initial parameter determination model, the processor may determine the loss value based on the hemodynamic characteristics determined by the vascular fluid dynamic model (e.g., a reduced-order and/or reduced-dimensionality model of a three-dimensional vascular fluid dynamic model) using the property parameters output by the initial parameter determination model, and the corresponding gold standard of the hemodynamic characteristics. The loss value is then configured to update the parameters of the initial parameter determination model, completing the network training and resulting in a trained parameter determination model.

In some embodiments, the processor may input the determination result from the initial parameter determination model into the vascular fluid dynamic model to obtain an intermediate hemodynamic characteristic. Based on the gold standard of the hemodynamic characteristic and the intermediate hemodynamic characteristic, a target loss is constructed. The target loss may be configured to represent an error between the gold standard of the hemodynamic characteristic and the intermediate. As used herein, the intermediate refers to an estimated value based on the vascular fluid dynamic model. The gold standard refers to a reference value. The model parameters of the initial parameter determination model may be adjusted based on the target loss until the target loss function satisfies a termination condition, resulting in the trained parameter determination model.

As an example, when the determination result includes property parameters for a plurality of vascular point samples, the processor may input the property parameters for each vascular point sample into the vascular fluid dynamic model to obtain the intermediate hemodynamic characteristics for each vascular point sample. Subsequently, the processor may input these intermediate hemodynamic characteristics along with the corresponding gold standard of the hemodynamic characteristics into a preset loss function to determine the target loss. Based on the target loss, the initial parameter determination model may undergo iterative training, ultimately generating a trained parameter determination model. The preset loss function may be constructed based on the intermediate hemodynamic characteristic and the gold standard hemodynamic characteristic for each vascular point sample. The process of inputting property parameters for each vascular point sample into the vascular fluid dynamic model to obtain the intermediate hemodynamic characteristics may be performed according to operation 430 in FIG. 4 and/or operation S4 in FIG. 5.

The intermediate hemodynamic characteristics may be determined based on the property parameters output by the initial parameter determination model during the training process.

After each iteration of optimizing the initial parameter determination model, the optimized parameter determination model may be configured to re-identify the property parameters at the vascular point samples. The vascular fluid dynamic model may be used to determine new hemodynamic characteristics for these vascular point samples based on the re-identified property parameters. These new hemodynamic characteristics may be compared to the gold standard of the hemodynamic characteristics for the vascular point samples, resulting in a new target loss. The optimized parameter determination model may be further refined based on the new target loss, enabling iterative training of the parameter determination model.

In some embodiments, when the cross-section corresponding to a vascular point sample is circular, the property parameters output by the initial parameter determination model for the vascular point sample may be closer to the true morphological parameters of the cross-section. Therefore, if the cross-section corresponding to a vascular point sample is circular, the first label for the vascular point sample may include the true morphological parameters of the cross-section. During iterative optimization of the initial parameter determination model, a first loss may be determined based on the intermediate hemodynamic characteristics and the gold standard of the hemodynamic characteristics, and a second loss may be determined based on the determination results of the initial parameter determination model and the true morphological parameters. The loss value for the vascular point sample may be determined based on the first loss and second loss. For example, the average of the first loss and second loss may be used as the loss value for the vascular point sample.

By including the true morphological parameters of the cross-section as additional labels when the vascular point samples correspond to circular cross-sections, the model training process becomes more accurate and may converge faster.

In some embodiments, one iteration of training may correspond to one or more vascular point samples. When a plurality of vascular point samples are involved in a single iteration, a loss value may be determined for each vascular point sample, and the average of these loss values may be taken as the target loss for that iteration.

During the iterative training process of the model, the model's weights may be continuously optimized to minimize the target loss, and the model parameters of the initial parameter determination model may be adjusted based on the target loss. The model training may be completed when the target loss of the initial parameter determination model satisfies the termination condition, resulting in a trained parameter determination model. In some embodiments, the termination condition may include convergence of the target loss or reaching a threshold number of iterations.

In some embodiments of the present disclosure, the hemodynamic characteristics are configured to guide the training of the parameter determination model, rather than relying solely on the gold standard corresponding to the property parameters outputted by the parameter determination model for model training which addresses the challenges associated with obtaining gold standard of the property parameters for abnormal vascular structures, which may be difficult to obtain and may lead to increased training difficulty and suboptimal training results.

In some embodiments, the property parameters output by the parameter determination model may be configured to determine at least two types of hemodynamic characteristics. For example, the parameter determination model may output a single numerical value for the equivalent diameter of a vascular point, which may be configured to determine both blood pressure and flow velocity. During the training of such a parameter determination model, both intermediate hemodynamic characteristics and gold standard of the hemodynamic characteristics include a plurality of parameter types, and each parameter type may be assigned a weight.

In some embodiments, the intermediate hemodynamic characteristic includes at least two first parameters, each of the at least two first parameters corresponds to a parameter type corresponding to a weight, and the gold standard hemodynamic characteristic includes at least two second parameters each of which corresponds to one of the at least two first parameters. By adjusting the weights of different parameter types during the training process, the focus of the property parameters output by the parameter determination model may be shifted towards evaluating the hemodynamic characteristic of the parameter type with the higher weight.

To train the type of parameter determination model, the processor may determine the loss value for each parameter type based on the gold standard of the hemodynamic characteristic and the intermediate hemodynamic characteristic corresponding to that type. The target loss is then determined based on the loss values and weights of the different parameter types. In some embodiments, for each of the at least two first parameters, the processor may determine a loss value corresponding to the first parameter based on the first parameter, the corresponding second parameter, and the corresponding weight; and determine the target loss based on loss values corresponding to the at least two first parameters. The first parameter is a parameter of one of the intermediate hemodynamic characteristics comprising at least two parameter types. The intermediate hemodynamic characteristic is a parameter of one of the gold standard hemodynamic characteristics comprising at least two parameter types.

For example, loss values for blood pressure (loss 1), flow velocity (loss 2), wall shear stress (loss 3), and circumferential stress (loss 4) may be determined based on their respective gold standards and intermediate values. The overall loss value L is then determined as L=loss 1W1+loss 2W2+loss 3W3+loss 4W4, where W1, W2, W3, and W4 are the weights assigned to the blood pressure, the flow velocity, the wall shear stress, and the circumferential stress, respectively.

In some embodiments, the processor may assign different weights to different parameter types. These weights may be determined in various ways. For example, the processor may obtain the weights based on user input. If the user focuses on determining the blood pressure, the weight for the blood pressure (loss 1) may be increased. Similarly, if the user is interested in the blood pressure and the flow velocity, the weights for the blood pressure (loss 1) and the flow velocity (loss 2) may be increased.

Some embodiments of the present disclosure allow for the training of a parameter determination model that outputs a single property parameter (e.g., equivalent diameter), which may be configured to determine hemodynamic characteristics of different types, which simplifies the process by eliminating the need to train a plurality of models or determine a plurality of property parameters for each hemodynamic characteristic. It enhances the efficiency of hemodynamic characteristic evaluation. Additionally, by adjusting the weights assigned to different hemodynamic characteristic types during model training, the outputted property parameters may be tailored to better suit specific needs, such as determining a particular hemodynamic characteristic more accurately.

In some embodiments, during the initial training of the parameter determination model, equal weights may be assigned to different types of hemodynamic characteristics. However, in practical applications, the weights may be adjusted based on specific requirements (e.g., emphasizing a particular type of hemodynamic characteristic), and the initial parameter determination model may be updated accordingly. For example, if the practical application focuses on determining the blood pressure, the initial parameter determination model may be updated by increasing the weight assigned to the blood pressure during training or by using training samples specifically related to the blood pressure.

By pre-training a generally applicable initial parameter determination model, some embodiments of the present disclosure improve the training efficiency of the parameter determination model, enabling faster convergence to target values. The significantly enhances the speed of parameter determination model training and reduces training difficulty. Furthermore, the ability to adjust weights or select specific types of hemodynamic characteristics based on actual needs allows for fine-tuning of the parameter determination model in subsequent applications, increasing its versatility and applicability.

In the training process described above for the parameter determination model, the error between the gold standard hemodynamic characteristics and the intermediate hemodynamic characteristics output by the vascular fluid dynamic model is used as the loss (or cost function) for model training, which circumvents the challenge of obtaining gold standard property parameters for vascular structures, which may be difficult to acquire. However, it introduces a longer chain of loss determination compared to directly training the parameter determination model based on the output of a gold training network and corresponding gold standards.

To further improve the training efficiency of obtaining the parameter determination model and make the network converge faster, in some embodiments, the processor may also pre-train the preset neural network first. Then, using the method described in FIG. 4 above, the preset neural network is trained until it meets the preset a training condition to obtain a well-trained parameter determination model. In this embodiment, the initial parameter determination model may be a pre-trained machine learning model.

In some embodiments, the processor may obtain a preset neural network and pre-train the preset neural network to obtain the initial parameter determination model.

In some embodiments, the preset neural network may be a machine learning model that has not undergone any training. In some embodiments, after constructing a basic model structure the processor may obtain the preset neural network through parameter initialization. In some embodiments, the processor may initialize parameters by assigning zero, unity, or random values to the model parameters of the preset neural network. In some embodiments, the processor may assign random values to the model parameters of the preset neural network through random initialization methods such as Gaussian distribution initialization or uniform distribution initialization.

In some embodiments, the processor may use the property parameters of the vascular structure sample in the first medical image sample as the gold standard of the property parameters. Based on the first medical image samples and the corresponding gold standard of the property parameters, the preset neural network may be trained to obtain the initial parameter determination model.

In some embodiments, the processor may train and update model parameters of the preset neural network based on a plurality of second training samples with second labels, using various methods to obtain the initial parameter determination model. For example, training of the preset neural network may be performed based on the gradient descent algorithm. As an example, the plurality of second training samples with second labels may be input into the preset neural network, and a loss function may be constructed based on the second labels and the output results of the preset neural network. The model parameters of the preset neural network may be iteratively updated based on the loss function. During the training process, the preset neural network may be continuously optimized by adjusting weights of the preset neural network to minimize the loss function. When the loss function of the preset neural network satisfies a preset condition, the model training of the preset neural network is completed, and a trained initial parameter determination model may be obtained. The preset condition may include convergence of the loss function, the number of iterations reaching a threshold, etc.

In some embodiments, the second training samples may include second medical image samples, and the second labels may be property parameters of the vascular structure sample in the second medical image samples. For example, a second label corresponding to a second medical image sample may be the one or more property parameters corresponding to each vascular point sample in the vascular structure sample of the second medical image sample. In some embodiments, the definition of the second training samples may be similar to the definition of the first training samples. The second training samples may be the same as or different from the first training samples.

In some embodiments, the processor may obtain values of the property parameters manually annotated by users or annotated based on experience as the gold standard of the property parameters (i.e., the second labels). Based on these gold standard of the property parameters, the preset neural network may be pre-trained for a period of time to obtain the aforementioned initial parameter determination model.

For example, if a vascular point sample corresponds to a circular cross-section, the second label may be the actual diameter, the actual radius, the actual cross-sectional area, or the actual cross-sectional perimeter of the cross-section. As another example, if a vascular point sample corresponds to a non-circular cross-section, the second label may be the hydraulic diameter, the actual cross-sectional area, or the actual cross-sectional perimeter of the cross-section.

In some embodiments, if a vascular point sample corresponds to a circular cross-section, the property parameters output by the parameter determination model are closer to the actual morphological parameters corresponding to the cross-section. Therefore, to improve the accuracy of pre-training, the vascular point samples in the second training samples may all be vascular point samples corresponding to circular cross-sections. In the way, the corresponding second labels may be more accurate, and the pre-training process may also be more accurate.

In some embodiments, the model training process (e.g., processes 600-900) and process 400 for evaluating a hemodynamic characteristic (the model application process) may be executed by the same or different executors. For example, the model training process is executed by the processing equipment of the model supplier, while the process 400 for evaluating a hemodynamic characteristic is executed by the processing equipment (e.g., the processor 120) at the model application end (e.g., a hospital).

Although manually annotated gold standard property parameters may not be entirely accurate, especially in terms of satisfying the requirements of vascular fluid mechanics model calculations, they may provide a certain degree of approximation. Through pre-training, the neural network may better fit the manually annotated gold standard of the property parameters. Then, by transferring the neural network to the aforementioned training process, the vascular fluid dynamics model parameters are fitted according to the fluid dynamics results of the vascular fluid dynamic model, making the neural network to converge to the target value faster and better, greatly improving the training speed of the preset recognition network and further reducing the difficulty of network training.

FIG. 7 is a flowchart illustrating an exemplary process for obtaining a gold standard of a hemodynamic characteristic according to some embodiments of the present disclosure. In some embodiments, the process 700 for determining a gold standard of a hemodynamic characteristic may be executed by a processor (e.g., the processor 120 or other processing devices). For example, process 700 may be stored in the form of programs or instructions in a storage device (e.g., the storage device 130), and when the processor executes these programs or instructions, process 700 may be implemented. The schematic diagram of the operations of process 700 presented below is illustrative. In some embodiments, the process may be completed with one or more additional operations not described and/or one or more operations not discussed. Additionally, the order of the operations of process 700 shown in FIG. 7 and described below is not restrictive. Obtaining the gold standard of the hemodynamic characteristic in operation 610 of FIG. 6 may be executed according to process 700. As shown in FIG. 7, process 700 includes the following operations.

In 710, the processor 120 (for example, the training module 350) may generate a three-dimensional vascular model of a vascular structure sample based on the vascular structure sample in a first medical image sample.

In some embodiments, the processor may extract the vascular structure sample from the first medical image sample.

In some embodiments, the processor may create the three-dimensional vascular model of the vascular structure sample through various methods based on the vascular structure sample. For example, the processor may perform three-dimensional reconstruction on the vascular structure sample based on an image reconstruction algorithm to obtain the three-dimensional vascular model of the vascular structure sample. Exemplary image reconstruction algorithms may include an edge detection algorithm, a curve fitting algorithm, a similarity algorithm, etc. As another example, the processor may label a plurality of vascular points in the vascular structure sample and generate the three-dimensional vascular model of the vascular structure sample based on the positions of the vascular points and the equivalent radii of blood vessels at those points. For more information about the vascular points, please refer to FIG. 4 and related descriptions.

In some embodiments, the processor may perform mesh generation on the three-dimensional vascular model to obtain a meshed three-dimensional vascular model by dividing the three-dimensional vascular model into a plurality of polygonal meshes. The vertices of these polygonal meshes may be designated as mesh vertices. These polygonal meshes may be triangles, quadrilaterals, or other polygons.

In 720, the processor 120 (for example, the training module 350) may determine a three-dimensional fluid dynamic parameter corresponding to each of the plurality of mesh vertices based on the first medical image sample, the three-dimensional vascular model, and a three-dimensional vascular fluid dynamic model.

The three-dimensional vascular fluid dynamic model is a model configured to determine a three-dimensional fluid dynamic parameter corresponding to each mesh vertex in the three-dimensional vascular model. In some embodiments, the processor may establish the three-dimensional vascular fluid dynamic model based on a continuity equation, Bernoulli's equation, a momentum equation, etc., combined with preset boundary conditions and related parameter settings.

In some embodiments, an input of the three-dimensional vascular fluid dynamic model includes the first medical image sample and the meshed three-dimensional vascular model, and an output of the three-dimensional vascular fluid dynamic model is the three-dimensional fluid dynamic parameter corresponding to each mesh vertex in the meshed three-dimensional vascular model.

The three-dimensional fluid dynamic parameter refers to a parameter related to a blood flow in a three-dimensional space. The parameter includes at least one of a pressure, a pressure drop, a flow velocity, a wall shear stress, a circumferential stress, a fractional flow reserve, a blood flow volume, a blood viscosity, and a blood flow pattern at each mesh vertex.

In some embodiments, the output of the three-dimensional vascular fluid dynamic model is related to a boundary condition and a related parameter. When performing calculations based on the three-dimensional vascular fluid dynamic model, the processor may adopt a CFD technique. Depending on the boundary conditions and related parameter, various three-dimensional fluid dynamic parameters may be determined. For example, the processor may set boundary conditions such as pressure and flow rate, and solve a discretized differential equation set based on the Navier-Stokes equation to obtain three-dimensional fluid dynamic parameters such as blood pressure and flow velocity.

In 730, the processor 120 (for example, the training module 350) may determine the gold standard of the hemodynamic characteristic based on the three-dimensional fluid dynamic parameter.

In some embodiments, the processor may map a three-dimensional CFD result (i.e., the three-dimensional fluid dynamic parameter) to one dimension, thereby obtaining a one-dimensional CFD result. The allows for the training of the initial parameter determination model based on the gold standard of the hemodynamic characteristic in the form of one-dimensional CFD, resulting in a trained parameter determination model.

In some embodiments, the processor may map the three-dimensional CFD result to the one-dimensional CFD result in various ways. For example, the processor may use dimension shuffling transformation to map the three-dimensional CFD result to the one-dimensional CFD result. As another example, the processor may map the mesh vertices in the three-dimensional vascular model to the centerline of the vascular structure, thus mapping the three-dimensional CFD result to the one-dimensional CFD result.

Illustratively, when mapping the three-dimensional CFD result to the one-dimensional CFD result, the hemodynamic characteristics of various three-dimensional meshes on the vascular structure are mapped to the corresponding hemodynamic characteristics of various vascular point samples on the vascular centerline corresponding to the vascular structure sample. Based on the, while using the three-dimensional vascular fluid dynamic model to evaluate the hemodynamic characteristics of the vascular structure in the first medical image sample, the processor also needs to determine various vascular point samples on the vascular centerline corresponding to the vascular structure sample in the first medical image sample. Further, based on the results of blood flow evaluation, that is, the hemodynamic characteristics of various three-dimensional meshes on the vascular structure sample, the processor determines the hemodynamic characteristics corresponding to various vascular point samples on the vascular centerline. For more information about the mapping the three-dimensional CFD result to one dimension to determine the one-dimensional gold standard of the hemodynamic characteristic, please refer to FIG. 8 and its related descriptions.

FIG. 8 is a flowchart illustrating an exemplary process for determining a gold standard hemodynamic characteristic according to other embodiments of the present disclosure. In some embodiments, the method for determining a gold standard hemodynamic characteristic may be executed by a processor (e.g., the processor 120 or other processing devices). For example, process 800 may be stored in a storage device (e.g., the storage device 130) in the form of a program or instructions. When the processor executes the program or instructions, process 800 may be achieved. The schematic diagram of the operations of process 800 presented below is illustrative. In some embodiments, the process may be completed with one or more additional operations not described and/or one or more operations not discussed. Furthermore, the order of the operations of process 800 shown in FIG. 8 and described below is not restrictive. Process 600 in FIG. 6 and/or process 700 in FIG. 7 may be executed according to process 800. As shown in FIG. 8, process 800 includes the following operations.

In 810, the processor 120 (e.g., the training module 350) may extract a centerline of a vascular structure sample based on a three-dimensional vascular model.

The centerline (also referred to as vascular centerline) of the vascular structure sample refers to a line connecting points in the vascular structure sample that are equidistant (or approximately equidistant) from the vascular wall. In some embodiments, the processor may extract the vascular centerline of the vascular structure sample in various ways. For example, the processor may use a vascular centerline extraction algorithm to extract the vascular centerline of the vascular structure sample. As another example, the processor may perform vascular segmentation on the first medical image sample to obtain a vascular segmentation mask, and then determine the vascular centerline based on the vascular segmentation mask.

In 820, the processor 120 (e.g., the training module 350) may determine a plurality of target centerline points on the centerline.

In some embodiments, the processor may map the vascular point samples determined in the first medical image sample to the vascular centerline of the three-dimensional vascular model to obtain the target centerline points corresponding to the vascular point samples.

For more information about the vascular points, please refer to FIG. 4 and related descriptions.

In some embodiments, the processor may determine positional information of each target centerline point on the vascular centerline.

In 830, for each of the plurality of target centerline points, the processor 120 (e.g., the training module 350) may obtain at least one neighboring mesh vertex within a preset range of the target centerline point.

The neighboring mesh vertex refers to a mesh vertex within the preset range of the target centerline point. For more information about the mesh vertices, please refer to FIG. 7 and related descriptions.

In some embodiments, the preset range of the target centerline point may be a region within a certain distance around the target centerline point. For example, the preset range may be within a distance of no more than 3 mm from the target centerline point.

In some embodiments, the preset range may be set in advance. In other embodiments, the processor may adjust the preset range based on an actual condition. For example, when the target centerline point is located at a vascular bifurcation or stenosis, the processor may appropriately reduce the preset range to improve the accuracy of candidate fluid dynamics parameters.

In some embodiments, for each target centerline point among a plurality of target centerline points, the processor may input the positional information of the target centerline point into the three-dimensional vascular fluid dynamic model to obtain at least one neighboring mesh vertex within the preset range of the target centerline point.

In 840, the processor 120 (e.g., the training module 350) may determine a candidate fluid dynamic parameter corresponding to the target centerline point based on the three-dimensional fluid dynamic parameter corresponding to the at least one neighboring mesh vertex.

In some embodiments, the processor may determine the three-dimensional fluid dynamic parameter corresponding to the neighboring mesh vertex based on the three-dimensional fluid dynamics model. In some embodiments, the processor may input the positional information of the neighboring mesh vertex into the three-dimensional fluid dynamic model to determine the corresponding three-dimensional fluid dynamics parameter.

In other embodiments, for each target centerline point among the plurality of target centerline points, the processor may input the positional information of the target centerline point into the three-dimensional vascular fluid dynamic model and directly determine the three-dimensional fluid dynamic parameters of a plurality of neighboring mesh vertices within the preset range of the target centerline point on the vascular structure.

The candidate fluid dynamic parameter refers to a fluid dynamic parameter configured to determine the gold standard of the hemodynamic characteristic.

In some embodiments, the processor may determine the candidate fluid dynamic parameter corresponding to the target centerline point through various methods. For example, the processor may determine the three-dimensional fluid dynamic parameter corresponding to the nearest neighboring mesh vertex to the target centerline point as the candidate fluid dynamic parameter for the target centerline point. As another example, the processor may determine one value among the mean, mode, or median of the three-dimensional fluid dynamic parameter corresponding to the at least one neighboring mesh vertex, and determine the value as the candidate fluid dynamic parameter for the target centerline point. By way of example only, the average blood pressure of a plurality of neighboring mesh vertices within the preset range of the target centerline point may be used as the gold standard of the blood pressure corresponding to that target centerline point.

As another example, the processor may perform a weighted sum of the three-dimensional fluid dynamic parameter corresponding to the at least one neighboring mesh vertex based on a distance between each of the at least one neighboring mesh vertex and the target centerline point, and determine the weighted sum as the candidate fluid dynamic parameter for the target centerline point. In the case, the closer the neighboring mesh vertex is to the target centerline point is, the greater the weight of the corresponding three-dimensional fluid dynamic parameter is.

Through the above methods, the gold standard of the hemodynamic characteristic corresponding to each vascular point on the vascular centerline may be determined, thereby mapping the three-dimensional CFD result corresponding to the three-dimensional vascular model to the vascular evaluation result (i.e., one-dimensional CFD result) corresponding to the one-dimensional vascular model.

In 850, the processor 120 (e.g., the training module 350) may determine the gold standard hemodynamic characteristic based on the candidate fluid dynamic parameters corresponding to the plurality of target centerline points.

Since the target centerline points are points obtained by mapping vascular points to a one-dimensional vascular centerline model, the candidate fluid dynamic parameters corresponding to each target centerline point may be determined as the gold standard hemodynamic characteristic for the corresponding vascular point.

Based on the, for each vascular point on the vascular centerline, the processor may input the image of the region of interest corresponding to the vascular point into the initial parameter determination model for determination to obtain the property parameter of the vascular point. Then, the property parameter of the vascular point is input into the one-dimensional vascular fluid dynamic model to generate the intermediate hemodynamic characteristic corresponding to the vascular point. By comparing the intermediate hemodynamic characteristic with the gold standard of the hemodynamic characteristic corresponding to the same vascular point, the target loss associated with the initial parameter determination model may be determined. The target loss may then be configured to train the initial parameter determination model.

It should be noted that during each iteration of determining the target loss, the target loss may be determined based on one or plurality of vascular points on the vascular centerline. The present disclosure does not impose specific limitations in this regard.

In other embodiments, when determining the gold standards of the hemodynamic characteristic corresponding to vascular points on the vascular centerline, the computer device may establish a three-dimensional vascular fluid dynamic model based on the vascular structure in the first medical image sample. The vascular centerline of the vascular structure is identified in the first medical image sample to obtain positional information of the target centerline points of the vascular structure. Subsequently, the positional information of the target center points is input into the three-dimensional vascular fluid dynamic model to generate the gold standards of the hemodynamic characteristic corresponding to the target centerline points.

In some embodiments of the present disclosure, the gold standard of the hemodynamic characteristic for each vascular point is determined by mapping the three-dimensional vascular fluid dynamic model to one dimension, which is equivalent to using precise the three-dimensional CFD result as the gold standard for training to obtain a parameter determination model and a one-dimensional vascular fluid dynamic model. As a result, when the one-dimensional vascular fluid dynamic model uses the property parameter output by the parameter determination model for hemodynamic characteristic evaluation, it may achieve hemodynamic effects equivalent to three-dimensional CFD results. Therefore, adopting the method may improve the training effectiveness of the parameter determination model, enhance the accuracy of the preset recognition network, and consequently improve the accuracy of blood flow evaluation. In later applications, the use of a high-precision parameter determination model and a one-dimensional vascular fluid dynamic model enables accurate hemodynamic evaluation of blood vessels with any abnormal structure. The not only simplifies the computational complexity of blood flow evaluation but also enhances the accuracy and efficiency of blood flow evaluation.

FIG. 9 is a schematic diagram illustrating an exemplary process for training a parameter determination model according to some embodiments of the present disclosure.

Taking the equivalent diameter as an example of a property parameter, in the embodiment, a parameter determination model for determining the equivalent diameter D is trained by coupling a neural network model with a one-dimensional vascular fluid dynamic model. The equivalent diameter for each vascular point on the vascular centerline may be determined using the trained neural network model. Illustratively, the neural network model may consist of a convolutional neural network and a fully connected network, or it may be composed of a transformer self-attention network model, etc. An input of the neural network model is a local VOI image within a preset range of a vascular point, and an output of the neural network model is a corresponding property parameter of the vascular point, including but not limited to the equivalent diameter D.

As shown in FIG. 7, training the neural network model involves three parts: a three-dimensional vascular fluid dynamic model, a one-dimensional reduced-order vascular fluid dynamic model, and a neural network model.

The three-dimensional vascular fluid dynamic model is configured to generate three-dimensional fluid dynamic results corresponding to medical image samples. Illustratively, the processor may extract coronary arteries from the input medical image samples and establish a three-dimensional vascular model of the coronary arteries. Then, based on the three-dimensional vascular model of the coronary arteries, the three-dimensional vascular model of the coronary arteries may be meshed to obtain a three-dimensional grid, and a three-dimensional vascular fluid dynamic model may be constructed. Fluid dynamic parameters, i.e., three-dimensional fluid mechanics results, for each mesh vertex on the three-dimensional grid may be determined based on the three-dimensional vascular fluid dynamic model.

When evaluating a blood flow based on the three-dimensional vascular fluid dynamic model, a CFD technique may be employed. By inputting different boundary conditions and parameter settings, property parameters such as a pressure (a blood pressure), a flow velocity, a wall shear stress, and a circumferential stress may be determined.

Furthermore, to guide the output of the one-dimensional vascular fluid dynamic model using the three-dimensional fluid dynamic results, the three-dimensional fluid dynamic results may be mapped to one dimension. Illustratively, the processor may extract the vascular centerline from the medical image sample and determine one or more mesh vertices within a preset range for each vascular point on the vascular centerline. Based on the fluid dynamic parameters corresponding to these mesh vertices, the fluid dynamic parameters for the vascular points on the vascular centerline are determined to determine hemodynamic characteristics for the one-dimensional centerline fluid dynamic model, also known as the gold standard of hemodynamic characteristics.

Based on the aforementioned one-dimensional vascular fluid dynamic model, it can be seen that the pressure drop at vascular points may be determined based on the equivalent diameter, which means the blood pressure (or pressure) at the vascular points may be determined. Therefore, for the gold standard hemodynamic characteristics, the processor may determine the pressure corresponding to each vascular point on the vascular centerline based on the pressure associated with each mesh vertex, serving as the gold standard of the pressure value for each vascular point.

In some embodiments, the processor may extract local regions of interest images corresponding to each vascular point from the medical image samples and input the local regions of interest images into an initial deep learning network for equivalent diameter recognition, obtaining the initial equivalent diameter for each vascular point. Subsequently, the initial equivalent diameter for each vascular point is input into the aforementioned one-dimensional vascular fluid dynamic model to determine a pressure drop for each vascular point. The pressure at the starting point of the vascular and each pressure drop are configured to determine an intermediate pressure value for each vascular point. Furthermore, the target loss is determined based on the intermediate pressure values for each vascular point and the corresponding gold standard pressure values. Gradient backpropagation is performed based on the target loss to iteratively train the initial deep learning network until a termination condition is met, resulting in a trained deep learning network. At the point, the deep learning network possesses the functionality to recognize equivalent diameters.

It should be noted that when the deep learning network needs to recognize other property parameters, the aforementioned training process may also be employed. For a deep learning network, it may output one or plurality of different property parameters. Of course, different deep learning networks may also be configured to output different property parameters separately.

Illustratively, after training is completed, the testing phase may begin. During the testing phase, the test medical images undergo preprocessing, including but not limited to extracting local regions of interest images corresponding to various vascular points on each vascular centerline. Subsequently, these local regions of interest images are input into the trained deep learning network for determination to the equivalent diameter for each vascular point. The equivalent diameter for each vascular point is then input into the aforementioned one-dimensional vascular fluid dynamic model to obtain the pressure drop and pressure value for each vascular point.

In the example, a one-dimensional vascular fluid dynamic model simulating vascular fluid is established. Then, using precise results obtained from the three-dimensional vascular fluid dynamic model s as the training set, the d deep learning network for determining the equivalent diameter that used by the one-dimensional vascular fluid mechanics model is trained. The allows the one-dimensional vascular fluid dynamic model to achieve hemodynamic effects equivalent to three-dimensional CFD results when using the equivalent diameter for determining the hemodynamic characteristic. Additionally, by combining the iterative process of deep learning with the computational process of the one-dimensional vascular fluid dynamic model, the hemodynamic results of the three-dimensional CFD are used as the training set. The difference between the hemodynamic results of the one-dimensional vascular fluid dynamic model and the three-dimensional vascular fluid dynamic model serves as the cost function to train the deep learning network for determining the equivalent diameter that is used by the one-dimensional vascular fluid dynamic model for determining the hemodynamic characteristic.

Adopting the method preserves the simplicity and speed of the one-dimensional vascular fluid dynamic model while maintaining the accuracy of the three-dimensional vascular fluid dynamic model. The determination of the equivalent diameter, compared to the traditional determination of the hydraulic diameter, offers advantages for determining hemodynamic characteristics such as fraction flow reservation (FFR) based on coronary blood flow, resulting in more accurate blood flow evaluation outcomes.

FIG. 10 is an exemplary schematic diagram illustrating a parameter determination model according to some embodiments of the present disclosure.

In some embodiments, the hemodynamic characteristic includes at least two parameter types, and the parameter determination model may include at least two parameter determination sub-models. At least one property parameter outputted by each parameter determination sub-model is configured to determine at least one type of hemodynamic characteristic. For example, as shown in FIG. 10, the parameter determination model may include parameter determination sub-models 1, 2, . . . , n. A property parameter 1 output by the parameter determination sub-model 1 may be configured to evaluate a hemodynamic characteristic 1, a property parameter 2 output by the parameter determination sub-model 2 may be configured to evaluate a hemodynamic characteristic 2, . . . , and a property parameter n output by parameter determination sub-model n may be configured to evaluate a hemodynamic characteristic n. Hemodynamic characteristics 1, 2, . . . , n are different, but property parameters 1, 2, . . . , n may be the same or different.

As an example, the hemodynamic characteristic may include types such as blood pressure and flow rate. The parameter determination model may include a parameter determination sub-model for evaluating a blood pressure and a parameter determination sub-model for evaluating a flow rate. On or more property parameters outputted by the parameter determination sub-model for evaluating a blood pressure may be configured to determine a blood pressure, and one or more property parameters outputted by the parameter determination sub-model for evaluating a flow rate may determine a flow rate.

In some embodiments, the parameter determination model includes parameter determination sub-models designed to evaluate each type of hemodynamic characteristic among all parameter types. For example, if there are a total of n types of hemodynamic characteristics, the parameter determination model may include n parameter determination sub-models, each of the n parameter determination sub-models may be configured to evaluate one type of hemodynamic characteristic. The property parameters outputted by each parameter determination sub-model may be configured to evaluate one type of hemodynamic characteristic.

In some embodiments, the parameter determination model includes at least two input layers and at least two output layers, and each parameter determination sub-model consists of one input layer and one output layer. In certain embodiments, for the at least two parameter determination sub-models included in the parameter determination model, input data to the respective input layers of the at least two parameter determination sub-models may be the same, while output results of the output layers of the at least two parameter determination sub-models may be different. For example, when the parameter determination model includes the parameter determination sub-model for evaluating a blood pressure and the parameter determination sub-model for evaluating a flow rate, both the parameter determination sub-models receive the same medical image of the subject as input data, but the output layer of the parameter determination sub-model for evaluating a blood pressure outputs a blood pressure, and the output layer of the parameter determination sub-model for evaluating a flow rate output the flow rate.

In some embodiments, the parameter determination model includes one input layer and at least two output layers. The at least two parameter determination sub-models in the parameter determination model share the same input layer, but the at least two parameter determination sub-models output different property parameters through the output layers of the at least two parameter determination sub-models. For example, if the parameter determination model includes a parameter determination sub-model for evaluating a blood pressure and a parameter determination sub-model for evaluating a flow rate, the parameter determination sub-model for evaluating a blood pressure and the parameter determination sub-model for evaluating a flow rate may share the same input layer.

In certain embodiments, where each type of hemodynamic characteristic has a corresponding parameter determination sub-model, and plurality of parameter determination sub-models share the same input layer, the processor may preset the output layer of the parameter determination sub-model for evaluating the specific type of hemodynamic characteristic to run, while setting the output layers of the parameter determination sub-models for evaluating other hemodynamic characteristics to stop running, if the evaluation of a particular type of hemodynamic characteristic is needed.

In some embodiments, the at least two parameter determination sub-models included in the parameter determination model may be trained separately. During training, the processor may set different gold standards of the hemodynamic characteristic for different parameter determination sub-models. For example, when training a parameter determination sub-model for evaluating a blood pressure, the gold standard of the hemodynamic characteristic may be a gold standard of the blood pressure, and the intermediate hemodynamic characteristic may be an intermediate blood pressure. The training process for the parameter determination sub-model is identical to that described in FIG. 4A for the parameter determination model, with more details provided in the preceding description.

In certain embodiments, the training samples configured to train parameter determination sub-models corresponding to different parameter types may be the same or different. For example, a training sample 1 may be configured to train the parameter determination sub-model 1, whose outputted property parameter 1 is for evaluating the hemodynamic characteristic 1, while a training sample 2 is used for training the parameter determination sub-model 2, which outputs property parameter 2 for evaluating the hemodynamic characteristic 2. The training label 1 of training sample 1 corresponds to the hemodynamic characteristic 1, and the training label 2 of training sample 2 corresponds to the hemodynamic characteristic 2. Training samples 1 and 2 may be the same or different.

Merely by way of example, the parameter determination model may include parameter determination sub-models A-D. The parameter determination sub-models A may be configured to output a first equivalent diameter. The parameter determination sub-models B may be configured to output a first equivalent cross-sectional area. The first equivalent diameter and the first equivalent cross-sectional area may be configured to determine a first type of hemodynamic characteristic. The parameter determination sub-models C may be configured to output a second equivalent diameter. The parameter determination sub-models D may be configured to output a second equivalent cross-sectional area. The second equivalent diameter and the second equivalent cross-sectional area may be configured to determine a second type of hemodynamic characteristic.

In some embodiments of the present disclosure, by setting the parameter determination model to include plurality of parameter determination sub-models, each capable of outputting property parameters for evaluating at least one type of hemodynamic characteristic, the accuracy of hemodynamic characteristics determined based on these property parameters may be improved, meeting different practical needs.

Based on the same inventive concept, embodiments of the present disclosure also provide a computer-readable storage medium and a computer program product for implementing the method for evaluating a hemodynamic characteristic described above. The computer-readable storage medium stores a computer program that, when executed by a processor, implements the method for evaluating a hemodynamic characteristic in any of the embodiments described above. The computer program product includes a computer program that, when executed by a processor, implements the method for evaluating a hemodynamic characteristic in any of the embodiments described above.

It should be noted that the user information (including but not limited to user device information, personal information, etc.) and data (including but not limited to data for analysis, storage, display, etc.) involved in the present disclosure are all information and data authorized by the user or fully authorized by all parties, and the collection, use, and processing of relevant data need to comply with relevant regulations.

Those skilled in the art can understand that all or part of the processes in the method of the above embodiments may be completed by instructing relevant hardware through computer programs. The computer programs may be stored in a non-volatile computer-readable storage medium. When executed, the computer programs may include the processes of the embodiments of the above methods. Among them, any reference to memory, database, or other media used in the embodiments provided by the present disclosure may include at least one of non-volatile and volatile memory. The non-volatile memory may include read-only memory (ROM), magnetic tape, floppy disk, flash memory, optical storage, high-density embedded non-volatile memory, ReRAM, MRAM, FRAM, PCM, graphene memory, etc. The volatile memory may include random access memory (RAM) or external cache memory, etc. As an illustration and not a limitation, the RAM may take various forms such as static random access memory (SRAM) or dynamic random access memory (DRAM). The database involved in the embodiments provided by the present disclosure may include at least one of relational and non-relational databases. The non-relational database may include a blockchain-based distributed database, etc., but is not limited to this. The processor involved in the embodiments provided by the present disclosure may be a general processor, a central processor, a graphics processor, a digital signal processor, a programmable logic device, a quantum-based data processing logic device, etc., but is not limited to this.

It should be noted that the descriptions related to processes 400, 600, 700, and 800 mentioned above are solely for the purpose of illustration and explanation, and do not limit the scope of application of the present disclosure. For those skilled in the art, various modifications and changes can be made to processes 400, 600, 700, and 800 under the guidance of the present disclosure. However, these modifications and changes are still within the scope of the present disclosure.

The basic concepts have been described above, apparently, in detail, as will be described above, and does not constitute limitations of the disclosure. Although there is no clear explanation here, those skilled in the art may make various modifications, improvements, and modifications of present disclosure. This type of modification, improvement, and corrections are recommended in present disclosure, so the modification, improvement, and the amendment remain in the spirit and scope of the exemplary embodiment of the present disclosure.

At the same time, present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of present disclosure are not necessarily all referring to the same embodiment. Further, certain features, structures, or features of one or more embodiments of the present disclosure may be combined.

In addition, unless clearly stated in the claims, the order of processing elements and sequences, the use of numbers and letters, or the use of other names in the present disclosure are not configured to limit the order of the procedures and methods of the present disclosure. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, the disclosure does not mean that the present disclosure object requires more features than the features mentioned in the claims. Rather, claimed subject matter may lie in less than all features of a single foregoing disclosed embodiment.

In some embodiments, the numbers expressing quantities of ingredients, properties, and so forth, configured to describe and claim certain embodiments of the application are to be understood as being modified in some instances by the term “about,” “approximate,” or “substantially”. Unless otherwise stated, “about,” “approximate,” or “substantially” may indicate ±20% variation of the value it describes. Accordingly, in some embodiments, the numerical parameters used in the specification and claims are approximate values, and the approximation may change according to the characteristics required by the individual embodiments. In some embodiments, the numerical parameter should consider the prescribed effective digits and adopt a general digit retention method. Although in some embodiments, the numerical fields and parameters configured to confirm the breadth of its range are approximate values, in specific embodiments, such numerical values are set as accurately as possible within the feasible range.

With respect to each patent, patent application, patent application disclosure, and other material cited in the present disclosure, such as articles, books, manuals, publications, documents, etc., the entire contents thereof are hereby incorporated by reference into the present disclosure. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those described in the present disclosure, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A method for evaluating a hemodynamic characteristic implemented on a device including at least one processing device and at least one storage device, comprising:
obtaining a medical image of an object, the medical image including at least a portion of a vascular structure;
determining at least one property parameter of the vascular structure by inputting the medical image into a trained parameter determination model;
determining at least one hemodynamic characteristic of the vascular structure by inputting the at least one property parameter into a vascular fluid dynamic model.

2. The method of claim 1, wherein the at least one property parameter includes at least one of an equivalent diameter, an equivalent radius, an equivalent cross-sectional area, or an equivalent cross-sectional perimeter.

3-12. (canceled)

13. A system for evaluating a hemodynamic characteristic, comprising:
at least one storage device including a set of instructions; and
at least one processor in communication with the at least one storage device, wherein when executing the set of instructions, the at least one processor is directed to perform operations including:
obtaining a medical image of an object, the medical image including at least a portion of a vascular structure;
determining at least one property parameter of the vascular structure by inputting the medical image into a trained parameter determination model; and
determining at least one hemodynamic characteristic of the vascular structure by inputting the at least one property parameter into a vascular fluid dynamic model for a hemodynamic characteristic evaluation.

14. The system of claim 13, wherein the property parameter includes at least one of an equivalent diameter, an equivalent radius, an equivalent cross-sectional area, or an equivalent cross-sectional perimeter.

15. The system of claim 13, wherein the at least one hemodynamic characteristic includes at least one of a blood pressure, a flow velocity, a wall shear stress, a circumferential stress, a fractional flow reserve (FFR), a blood flow volume, a blood viscosity, or a blood flow morphology of the vascular structure.

16. The system of claim 13, wherein the vascular fluid dynamic model includes a reduced-order model of a three-dimensional vascular fluid dynamic model and/or a reduced-dimensionality model of the three-dimensional vascular fluid dynamic model.

17. The system of claim 13, wherein the trained parameter determination model is obtained through first operations including:
obtaining a first medical image sample and a corresponding gold standard hemodynamic characteristic, the gold standard hemodynamic characteristic is in a form of one-dimensional computational fluid dynamics (CFD);
determining a parameter determination result by inputting the first medical image sample into an initial parameter determination model;
obtaining the trained parameter determination model by training the initial parameter determination model based on the gold standard hemodynamic characteristic and the parameter determination result.

18. The system of claim 17, wherein obtaining the trained parameter determination model by training the initial parameter determination model based on the gold standard hemodynamic characteristic and the parameter determination result includes:
determining an intermediate hemodynamic characteristic by inputting the parameter determination result into the vascular fluid dynamic model;
determining a target loss based on the gold standard hemodynamic characteristic and the intermediate hemodynamic characteristic; and
obtaining the trained parameter determination model by adjusting, based on the target loss, a parameter of the initial parameter determination model until a termination condition is met.

19. The system of claim 18, wherein the intermediate hemodynamic characteristic includes at least two first parameters, each of the at least two first parameters corresponds to a parameter type corresponding to a weight, and the gold standard hemodynamic characteristic includes at least two second parameters each of which corresponds to one of the at least two first parameters; and
determining the target loss based on the gold standard hemodynamic characteristic and the intermediate hemodynamic characteristic includes:
for each of the at least two first parameters, determining a loss value based on the first parameter, the corresponding second parameter, and the corresponding weight; and
determining the target loss based on the loss values.

20. The system of claim 18, wherein obtaining the gold standard hemodynamic characteristic includes:
generating a three-dimensional vascular model of the vascular structure sample based on the vascular structure sample in the first medical image sample, the three-dimensional vascular model including multiple mesh vertices;
determining a three-dimensional fluid dynamic parameter corresponding to each of the plurality of mesh vertices based on the first medical image sample, the three-dimensional vascular model, and a three-dimensional vascular fluid dynamic model; and
determining the gold standard hemodynamic characteristic based on the three-dimensional fluid dynamic parameter by mapping the three-dimensional fluid dynamic parameter to one dimension.

21. The system of claim 20, wherein determining the gold standard hemodynamic characteristic based on the three-dimensional fluid dynamic parameter includes:
extracting a centerline of the vascular structure sample based on the three-dimensional vascular model;
determining a plurality of target centerline points on the centerline;
for each of the plurality of target centerline points:
obtaining at least one neighboring mesh vertex within a preset range of the target centerline point; and
determining a candidate fluid dynamic parameter corresponding to the target centerline point based on the three-dimensional fluid dynamic parameter corresponding to the at least one neighboring mesh vertex; and
determining the gold standard hemodynamic characteristic based on the candidate fluid dynamic parameters corresponding to the multiple target centerline points.

22. The system of claim 17, wherein the initial parameter determination model is obtained by second operations including:
obtaining a second medical image sample and a corresponding gold standard property parameter; and
obtaining the initial parameter determination model by training, based on the second medical image sample and the corresponding gold standard property parameter, a preset neural network.

23. The system of claim 13, wherein the at least one processor is directed to perform the operations including:
displaying the at least one hemodynamic characteristic of the vascular structure on a display device.

24. The system of claim 13, wherein the at least one processor is directed to perform the operations including:
displaying the at least one property parameter of the vascular structure and/or the medical image of the subject on a display device.

25-26. (canceled)

27. A method for training a parameter determination model implemented on a device including at least one processing device and at least one storage device, wherein the parameter determination model is configured to determine a property parameter of a vascular structure in a medical image, and the property parameter is configured to determine a hemodynamic characteristic of the vascular structure based on a vascular fluid dynamic model, the method comprising:
obtaining a first medical image sample and a corresponding gold standard hemodynamic characteristic;
determining a parameter determination result by inputting the first medical image sample into an initial parameter determination model;
obtaining the trained parameter determination model based on the gold standard hemodynamic characteristic and the parameter determination result.

28. The method of claim 27, wherein obtaining the trained parameter determination model by training the initial parameter determination model based on the gold standard hemodynamic characteristic and the parameter determination result includes:
determining an intermediate hemodynamic characteristic by inputting the parameter determination result into the vascular fluid dynamic model;
determining a target loss based on the gold standard hemodynamic characteristic and the intermediate hemodynamic characteristic; and
obtaining the trained parameter determination model by adjusting, based on the target loss, a parameter of the initial parameter determination model until a termination condition is satisfied.

29. The method of claim 28, wherein
the intermediate hemodynamic characteristic includes at least two first parameters, each of the at least two first parameters corresponds to a parameter type corresponding to a weight, and the gold standard hemodynamic characteristic includes at least two second parameters each of which corresponds to one of the at least two first parameters; and
determining the target loss based on the gold standard hemodynamic characteristic and the intermediate hemodynamic characteristic includes:
for each of the at least two first parameters, determining a loss value based on the first parameter, the corresponding second parameter, and the corresponding weight; and
determining the target loss based on the loss values.

30. The methods of claim 27, wherein obtaining the gold standard hemodynamic characteristic corresponding to the first medical image sample includes:
generating a three-dimensional vascular model of the vascular structure sample based on the vascular structure sample in the first medical image sample, the three-dimensional vascular model including a plurality of mesh vertices;
determining a three-dimensional fluid dynamic parameter corresponding to each of the plurality of mesh vertices based on the first medical image sample, the three-dimensional vascular model, and a three-dimensional vascular fluid dynamic model; and
determining the gold standard hemodynamic characteristic based on the three-dimensional fluid dynamic parameter.

31. The method of claim 30, wherein determining the gold standard hemodynamic characteristic based on the three-dimensional fluid dynamic parameter includes:
extracting a centerline of the vascular structure sample based on the three-dimensional vascular model;
determining a plurality of target centerline points on the centerline;
for each of the plurality of target centerline points:
obtaining at least one neighboring mesh vertex within a preset range of the target centerline point; and
determining a candidate fluid dynamic parameter corresponding to the target centerline point based on the three-dimensional fluid dynamic parameter corresponding to the at least one neighboring mesh vertex; and
determining the gold standard hemodynamic characteristic based on the candidate fluid dynamic parameters corresponding to the plurality of target centerline points.

32. The method of claim 27, wherein the initial parameter determination model is obtained by operations including:
obtaining a second medical image sample and a corresponding gold standard property parameter; and
obtaining the initial parameter determination model by training, based on the second medical image sample and the corresponding gold standard property parameter, a preset neural network.

33-35. (canceled)
